(19) United States
(12) Patent Application Publication
(10) Pub. No.: US 2026/0259221 A1
(43) Pub. Date: Sep. 3, 2026

(54) METHODS OF ANALYZING RECOMBINANT POLYCLONAL PROTEINS

(71) Applicant: GigaGen, Inc., San Carlos, CA (US)

(72) Inventors: Hongjun YUE, Palo Alto, CA (US); Rena Aviva MIZRAHI, Pacifica, CA (US); Ariel Reed NIEDECKEN, San Francisco, CA (US); Yao Yuan CHIANG, San Francisco, CA (US)

(21) Appl. No.: 19/537,353

(22) Filed: Feb. 11, 2026

Related U.S. Application Data
(63) Continuation of application No. PCT/US2024/042293, filed on Aug. 14, 2024.
(60) Provisional application No. 63/519,815, filed on Aug. 15, 2023.

(51) Int. Cl. G01N 33/68; A61K 39/00; C07K 16/082; C07K 16/108; C07K 16/1203; C07K 16/1275; C07K 16/18; C40B 30/00; G01N 30/86

(52) U.S. Cl. CPC G01N 33/6854; C07K 16/082; C07K 16/108; C07K 16/1242; C07K 16/1275; C07K 16/18; G01N 30/8675; A61K 2039/505; C07K 2317/21; C07K 2317/622; C07K 2317/92; C40B 30/00

(57) ABSTRACT

Provided herein is a method for analyzing and characterizing compositions comprising recombinant polyclonal proteins (ABPs). Also provided are use of the method related to production, manufacture, distribution, storage and therapeutic use of the ABPs.

### 1. CROSS REFERENCE TO RELATED APPLICATIONS

This application is a continuation of International Application No. PCT/US2024/042293, filed Aug. 14, 2024, which claims priority to U.S. Provisional Application No. 63/519,815, filed Aug. 15, 2023, each of which is incorporated in its entired by reference herein.

### 2. FIELD

Provided herein is a method of analyzing a library comprising at least 100 antigen binding proteins (ABPs). Also provided are various uses and applications of the method related to production, manufacture, distribution, storage and therapeutic use of ABPs.

### 3. BACKGROUND

Passive immunizations (McDonagh, 1966) offer protective strategies for immunodeficient individuals who do not respond to active vaccines. For example, intravenous immunoglobulin (IVIg) is a broad-spectrum polyclonal antibody therapy derived from the plasma of thousands of human donors. IVIg is used as an antibody replacement therapy for patients with humoral immune deficiencies (Lucas et al., 2010; Resnick et al., 2012). However, IVIg has a low titer of antibodies directed against many common pathogens, which leads to significant morbidity and mortality in immune deficient patients (Orange et al., 2010). To increase anti-pathogen titers, some groups have developed high-titer plasma-derived antibodies, often called hyperimmunes (Bozzo & Jorquera, 2017). Hyperimmunes are commonly derived from the plasma of donors soon after administration of active vaccines, such as HyperHEP B (Grifols), which has a high titer against Hepatitis B virus.

Hyperimmunes derived from donors recently administered active vaccines are excellent choices for passive immunization, but to scale such products commercially is a challenge (Kreil et al., 2012). Importantly, it can be difficult to identify strong responders who are willing to be vaccinated and donate plasma repeatedly. Therefore, hyperimmune manufacturing lots are necessarily derived from different sets of donors, resulting in lot-to-lot variability. The anti-pathogen titer varies significantly across hyperimmunes, from as low as 2- to 3-fold (Schampera et al., 2017) to as much as 50-fold (Kreil et al., 2012). In some cases, therefore, physicians may simply administer larger doses of IVIg (Polilli et al., 2012). Physicians and patients would benefit from more consistent, higher titer hyperimmunes that are easier to manufacture at large scale.

Many of these problems could be solved by generating multivalent hyperimmune globulins using recombinant DNA technology combined with a microfluidics and molecular genomics strategy. The general strategy was previously used to create recombinant multivalent hyperimmune globulins against severe acute respiratory syndrome coronavirus-2 (SARS-COV-2) or Zika virus, as described in PCT/US2020/030878 filed on Apr. 30, 2020; PCT/US2021/037232 filed on Jun. 14, 2021; and PCT/US2021/044523 filed on Aug. 4, 2021, which are incorporated by reference in their entireties. The multivalent hyperimmune globulins contain thousands of antigen binding proteins (ABPs) with binding specificity to therapeutic target antigens. The RPPs showed greater therapeutic efficacy against the viral infections compared to IVIg demonstrating their potential as new therapeutics.

In order to facilitate the broad therapeutic use of the library of antigen binding proteins (ABPs), it is critical to generate a high-quality composition with thousands of ABPs with substantial yields and to maintain their characteristics and qualities. Accordingly, there is a need to develop a method for testing quality and identity of a mixture comprising hundreds or thousands of unique ABPs.

### 4. SUMMARY

A library of ABPs is a complex product representing a novel therapeutic modality. Control of such a mixture is complicated. Some differences are expected between lots, and slight changes in the population distribution have been noted without any impact on the critical quality attributes (CQAs). It is important to manufacture this product consistently for example, by using the same seed train, regardless of batch size, to maintain a consistent cell age across batches. Additionally, the manufactured product needs to be analyzed to ensure the safety, identity, strength, quality and potency of the mixture.

Provided herein is a novel method for testing libraries of ABPs for assessing and ensuring the quality and identity of the ABPs. The method can be used in the process of production, distribution, storage and therapeutic use of the libraries.

Accordingly, one aspect of the present disclosure provides a method of analyzing a test library comprising antigen binding proteins (ABPs), comprising: measuring a distribution of charge states of the test library, wherein the test library comprises at least 100 ABPs; comparing the distribution of charge states with a reference distribution, wherein the reference distribution is a distribution of charge states of a reference library; and determining a quality of the test library based on the comparison.

In some embodiments, the distribution of charge states is measured in step (a) by cation exchange-High Performance Liquid Chromatography (CEX-HPLC). In some embodiments, the CEX-HPLC is performed with a pH gradient from a mobile phase A to a mobile phase B, wherein the mobile phase A has a low pH from pH 5 to pH 7 and the mobile phase B has a high pH from pH 9 to pH 11. In some embodiments, the mobile phase A has a low pH from pH 5 to pH 6 or from pH 5.5 to pH 6. In some embodiments, the mobile phase B has a high pH from pH 10 to 11 or from pH 10 to 10.5.

In some embodiments, the CEX-HPLC is performed with a column comprising a strong cation exchanger. In some embodiments, the CEX-HPLC is performed with a column comprising a weak cation exchanger.

In some embodiments, the CEX-HPLC is performed at a flow rate between 0.25 mL/min and 2 mL/min. In some embodiments, the CEX-HPLC is performed at a flow rate between 0.5 mL/min and 1.5 mL/min, between 0.75 mL/min and 1 mL/min, or between 0.75 mL/min and 0.85 mL/min. In some embodiments, the CEX-HPLC is performed at a flow rate of 0.5 mL/min, 0.75 mL/min or 1.0 mL/min.

In some embodiments, the CEX-HPLC is performed at 20-40° C., at 25-35° C., at 25° C., at 30° C., or at 35° C.

In some embodiments, the CEX-HPLC is performed with 5-100% B gradient, 10-100% B gradient, 15-100% B gradient or 20-100% B gradient.

In some embodiments, the CEX-HPLC is performed with 30-60 min gradient time. In some embodiments, the CEX-HPLC is performed with 30-60 min gradient time, 30-45 min gradient time, 30-40 min gradient time, or 35 min gradient time, 40 min gradient time, 45 min gradient time, 50 min gradient time, 55 min gradient time, or 60 min gradient time.

In some embodiments, the CEX-HPLC is performed with a column packed with a resin having a particle size smaller than 3 μm, smaller than 2.9 μm, smaller than 2.8 μm, smaller than 2.7 μm, smaller than 2.6 μm, or smaller than 2.5 μm. In some embodiments the CEX-HPLC is performed with a column packed with a resin that is a porous resin or a non-porous region.

In some embodiments, the reference distribution is measured by cation exchange-High Performance Liquid Chromatography (CEX-HPLC). In some embodiments, the reference distribution is measured by cation exchange-High Performance Liquid Chromatography (CEX-HPLC) under a condition identical to the condition of measuring the distribution of charge states in (a).

In some embodiments, the reference library comprises the same ABPs as the test library. In some embodiments, the test library and the reference library are produced from the same production cell line or a progeny thereof. In some embodiments, the reference library has been analyzed by sequencing. In some embodiments, the reference library is a batch different from the test library.

In some embodiments, the reference library comprises one antibody. In some embodiments, the reference library comprises a plurality of antibodies. In some embodiments, the reference library has been generated by mixing a plurality of monoclonal antibodies.

In some embodiments, the reference library comprises a subset of the at least 100 ABPs in the test library. In some embodiments, the test library and the reference library have been separately generated.

In some embodiments, the test library comprises at least 500 ABPs, at least 1000 ABPs, at least 2000 ABPs, at least 3000 ABPs, at least 4000 ABPs, at least 5000 ABPs, at least 6000 ABPs, at least 7000 ABPs, at least 8000 ABPs, at least 9000 ABPs, or at least 10,000 ABPs.

In some embodiments, in step (b), the distribution of charge states is compared with a reference distribution based on peak retention times. In some embodiments, in step (b), the distribution of charge states is compared with a reference distribution based on peak areas, peak heights, or peak numbers.

In some embodiments, in step (c), the test library is determined to have a better quality when its distribution of charge states is closer to the reference distribution. In some embodiments, peak sizes, peak retention times, or peak numbers are compared between the test library and the reference library. In some embodiments, peak sizes, peak retention times, and peak numbers are compared between the test library and the reference library. In some embodiments, in step (c), the test library is determined to have a good quality when its distribution of charge states is at least 50%, 60%, 70%, 80%, 90%, 96%, 97%, 98%, or 99% identical to the reference distribution in terms of peak sizes, peak retention times, or peak numbers.

In some embodiments, in step (c), the quality of the test library is determined further based on potency of the test library measured by ELISA. In some embodiments, in step (c), the quality of the test library is determined further based on analysis of the test library by size exclusion chromatography (SEC)-HPLC.

In some embodiments, the SEC-HPLC is performed with an SEC column comprising small particle resin, wherein the particle size is smaller than 2.9 μm, 2.8 μm, 2.7 μm, 2.6 μm, or 2.5 μm. In some embodiments, the SEC-HPLC is performed using a BEH stationary phase. In some embodiments, the SEC-HPLC is performed using a mobile phase with a pH between 6.7 and 7.3, between 6.8 and 7.2, between 6.9 and 7.1 or about 7.0. In some embodiments, the SEC-HPLC is performed with a mobile phase comprising a NaCl concentration between 450 mM and 550 mM, between 480 mM and 520 mM or about 500 mM.

In some embodiments, in step (c), the quality of the test library is determined further based on at least one of followings: (i) amino acid sequences of IgG1 and IgK framework, optionally verified with an LC-MS reduced peptide mapping method; (ii) a disulfide bond linkage between IgG1 and IgK constant region, optionally verified with an LC-MS non-reduced peptide mapping method; (iii) a size heterogeneity of the test library, optionally characterized by a multi-angle light scattering (MALS) technique; (iv) a melting temperature of the test library, optionally measured by differential scanning calorimetry (DSC); (v) a glass transition temperature of the test library, optionally measured by differential scanning calorimetry (DSC); (vi) released N-glycan analysis; (vii) total sialic acid quantification; and (viii) hemagglutination method.

In some embodiments, the test library is a pharmaceutical composition comprising the ABPs and a pharmaceutically acceptable excipient. In some embodiments, the test library is proteins isolated from the host cell culture. In some embodiments, the test library has been prepared by a process comprising: generation the at least 100 ABPs by culturing a production cell line; and purification of the at least 100 ABPs.

In some embodiments, the purification is performed by at least one of the following steps: (i) affinity chromatography, (ii) low pH virus inactivation, (iii) hydrophobic interaction chromatography or membrane filtration, (iv) multimodal anion exchange chromatography or membrane filtration, (v) multimodal cation exchange chromatography, (vi) anion exchange chromatography or membrane filtration, (vii) cation exchange chromatography, (viii) virus filtration, and (ix) ultrafiltration and/or diafiltration.

In some embodiments, the purification is performed by two, three, four, five, or all six of the following steps: (i) affinity chromatography, (ii) low pH virus inactivation, (iii) hydrophobic interaction chromatography or membrane filtration, (iv) multimodal anion exchange chromatography or membrane filtration, (v) multimodal cation exchange chromatography, (vi) anion exchange chromatography or membrane filtration, (vii) cation exchange chromatography, (viii) virus filtration, and (ix) ultrafiltration and/or diafiltration. In some embodiments, the purification is performed by two, three, four, five, six, seven, eight, or all nine of the following steps: (i) affinity chromatography, (ii) low pH virus inactivation, (iii) hydrophobic interaction chromatography or membrane filtration, (iv) multimodal anion exchange chromatography or membrane filtration, (v) multimodal cation exchange chromatography, (vi) anion exchange chromatography or membrane filtration, (vii) cation exchange chromatography, (viii) virus filtration, and (ix) ultrafiltration and/or diafiltration.

In some embodiments, the ABPs are antibodies. In some embodiments, the ABPs are antibodies specific to an antigen. In some embodiments, the antigen is a viral or bacterial antigen.

In some embodiments, the method further comprises preparing a pharmaceutical composing the test library. In some embodiments, the method further comprises selecting the test library for preparation of a pharmaceutical composition if the test library meets acceptance criteria.

In some embodiments, the test library meets acceptance criteria when the test library has charge distribution at least 50%, 60%, 70% %, 80%, 85%, 90%, 96%, 97%, 98%, or 99% identical to the reference distribution in terms of peak sizes, peak retention times, and/or peak numbers. In some embodiments, the acceptance criteria comprise one or more factors selected from:
a. SEC (size exclusion chromatography)-HPLC of the test library indicates that peaks corresponding to polyclonal antibodies represent more than 65%, 70%, 75%, 80%, 85%, 90%, 95%, 97%, 98%, or 99% of the entire peaks;
b. SEC (size exclusion chromatography)-HPLC of the test library indicates that peaks corresponding to high molecular weight (HMW) represent less than 5.0%, less than 4.0%, less than 3.0%, less than 2.0% or less than 1.0% of the entire peaks; and
c. SEC (size exclusion chromatography)-HPLC of the test library indicates that peaks corresponding to low molecular weight (LMW) represent less than 5.0%, less than 4.0%, less than 3.0%, less than 2.0% or less than 1.0% of the entire peaks.

In some embodiments, the acceptance criteria comprise one, two, or three factors selected from a to c.

In some embodiments, the method further comprises preparing a pharmaceutical composition composing the test library. In some embodiments, the pharmaceutical composition is prepared when the test library has been selected for meeting one or more factors in the acceptance criteria.

In another aspect, the present disclosure provides a pharmaceutical composition composing the test library and prepared by any of the methods.

Another aspect of the present disclosure relates to method of analyzing a test library comprising antigen binding proteins (ABPs), comprising: (a) measuring size heterogeneity of the test library, wherein the test library comprises at least 100 ABPs; (b) comparing peaks corresponding to high molecular weight species (HMW), polyclonal antibody monomers (pAb peaks), and low molecular weight species (LMW); and (c) determining a quality of the test library based on the comparison.

In some embodiments, the size heterogeneity of the test library is measured by Size Exclusion Chromatography (SEC)-HPLC.

In some embodiments, the SEC-HPLC is performed with an SEC column comprising a small particle resin, wherein the particle size is smaller than 2.9 μm, 2.8 μm, 2.7 μm, 2.6 μm, or 2.5 μm. In some embodiments, the SEC-HPLC is performed using a BEH stationary phase. In some embodiments, the SEC-HPLC is performed using a mobile phase with a pH between 6.7 and 7.3, between 6.8 and 7.2, between 6.9 and 7.1 or about 7.0. In some embodiments, the SEC-HPLC is performed with a mobile phase with a NaCl concentration between 450 mM and 550 mM, between 480 mM and 520 mM or about 500 mM. In some embodiments, the SEC-HPLC is performed at a temperature between 28° C. and 32° C., between 29° C. and 31° C. or at about 30° C.

In some embodiments, the test library meets acceptance criteria when: a. SEC (size exclusion chromatography)-HPLC of the test library indicates that peaks corresponding to polyclonal antibodies represent more than 65%, 70%, 75%, 80%, 85%, 90%, 95%, 97%, 98%, or 99% of the entire peaks; b. SEC (size exclusion chromatography)-HPLC of the test library indicates that peaks corresponding to high molecular weight (HMW) represent less than 5.0%, less than 4.0%, less than 3.0%, less than 2.0% or less than 1.0% of the entire peaks; and/or c. SEC (size exclusion chromatography)-HPLC of the test library indicates that peaks corresponding to low molecular weight (LMW) represent less than 5.0%, less than 4.0%, less than 3.0%, less than 2.0% or less than 1.0% of the entire peaks.

In some embodiments, the method further comprises preparing a pharmaceutical composition comprising the test library.

In another aspect, the present disclosure provides a pharmaceutical composition comprising the test library prepared by the method disclosed herein,

### 5. BRIEF DESCRIPTION OF THE DRAWINGS

FIG. 1 is the charge state of a library of ABPs with binding specifity to Hepatitis B virus antigen (rHBIG) obtained by bioinformatic analysis.

FIG. 2 illustrates charge-state of the IgGs depending on the buffer pH.

FIG. 3 shows HPLC chromatograms of the same rHBIG sample from three separate runs.

FIG. 4 provides HPLC chromatograms of rHBIG obtained with three different column lots-Proteomix SCX-NP1.7 (4.6×100 mm) columns: S/N 2A54701 (LN DW054), S/N 0A60382 (LN DW166), S/N 9A60383 (LN 430794).

FIG. 5 provides HPLC chromatograms of rHBIG obtained with three different flow rates (0.5 mL/min, 0.75 mL/min and 1.0 mL/min).

FIG. 6 provides HPLC chromatograms of rHBIG obtained with three different gradients (15-100% B, 5-100% B, and 10-100% B).

FIG. 7 provides HPLC chromatograms of rHBIG obtained with three different gradient times (35 min, 45 min and 60 min).

FIG. 8 provides HPLC chromatograms of anti-HBV plasma hyperimmune (HyperHEP), a library of ABPs with binding specifity to Hepatitis B virus antigen (rHBIG), a library of ABPs with binding specifity to CoV antigen (rCIG), or a recombinant monoclonal antibody (anti-CTLA-4).

FIG. 9 provides HPLC chromatograms of a library of ABPs with binding specifity to Hepatitis B virus antigen (rHBIG) and six individual antibodies in the library (PN-6103.02, 6104.02, 6105.02, 6115.02, 6116.02, 6117.02).

FIG. 10 provides HPLC chromatograms of rHBIG obtained using two different columns (Proteomix SCX NP1.7 4.6×100 mm and MabPac SCX-10 5 μm, 4.6×250 mm).

FIG. 11 provides SEC-HPLC chromatograms of rHBIG from two different lots (Tox DS and GMP DS).

FIG. 12 provides SEC-HPLC chromatograms of rHBIG in the forced degradation study.

FIG. 13 provides SEC-MALS results for rHBIG from two different lots (Tox DS and GMP DS).

FIG. 14 provides HPLC chromatograms of rHBIG Tox DS and GMP DS.

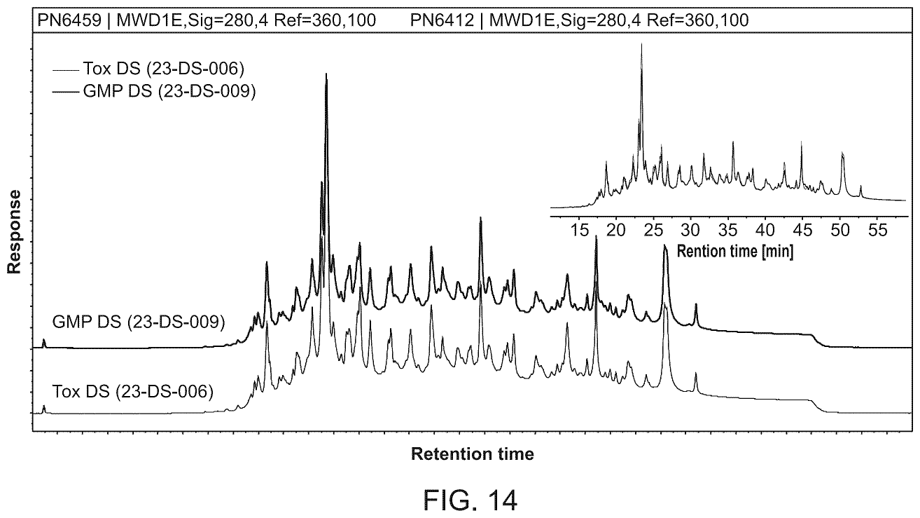

### 6. DETAILED DESCRIPTION

### 6.1. Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients.

The following terms, unless otherwise indicated, shall be understood to have the following meanings:

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies. One example of an antigen-binding domain is an antigen-binding domain formed by a V_{H}-V_{L} dimer.

The term “recombinant polyclonal antibody”, “recombinant polyclonal protein” or “RPP” refers to more than one recombinant antibodies, collectively comprising more than one antigen-binding domains that specifically bind to an antigen or epitope, or multiple antigens and epitopes. The recombinant polyclonal antibodies can be intact antibodies or variants or derivatives thereof. In some embodiments, the antigen-binding domains bind an antigen or epitope with specificity and affinity similar to that of a naturally occurring antibody. In some embodiments, a recombinant polyclonal antibody is a mixture of antibodies. In some embodiments, a recombinant polyclonal antibody comprises scFvs. In some embodiments, a recombinant polyclonal antibody comprises an alternative scaffold. In some embodiments, a recombinant polyclonal antibody consists of alternative scaffolds. In some embodiments, a recombinant polyclonal antibody consists essentially of alternative scaffolds. In some embodiments, a recombinant polyclonal antibody comprises an antibody fragment. In some embodiments, a recombinant polyclonal antibody consists of antibody fragments. In some embodiments, a recombinant polyclonal antibody consists essentially of antibody fragments.

The term “antigen-binding domain” means the portion of an antibody that is capable of specifically binding to an antigen or epitope.

The terms “full length antibody,” “intact antibody,” and “whole antibody” are used herein interchangeably to refer to an antibody having a structure substantially similar to a naturally occurring antibody structure and having heavy chains that comprise an Fc region.

The term “immunoglobulin” refers to a class of structurally related proteins, e.g., antibodies, generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated C_{L}.

An “antibody fragment” comprises a portion of an intact antibody, such as the antigen-binding or variable region of an intact antibody. Antibody fragments include, for example, Fv fragments, Fab fragments, F(ab′)₂ fragments, Fab′ fragments, scFv (sFv) fragments, and scFv-Fc fragments.

The term “antigen-binding protein” (ABP) refers to a protein comprising one or more antigen-binding domains that specifically bind to an antigen or epitope. In some embodiments, the antigen-binding domain binds the antigen or epitope with specificity and affinity similar to that of naturally occurring antibodies. In some embodiments, the ABP comprises an antibody. In some embodiments, the ABP consists of an antibody. In some embodiments, the ABP consists essentially of an antibody. In some embodiments, the ABP comprises an alternative scaffold. In some embodiments, the ABP consists of an alternative scaffold. In some embodiments, the ABP consists essentially of an alternative scaffold. In some embodiments, the ABP comprises an antibody fragment. In some embodiments, the ABP consists of an antibody fragment. In some embodiments, the ABP consists essentially of an antibody fragment.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “polyclonal antibody” refers to a mixture of at least two monoclonal antibodies. Polyclonal antibodies may be either monospecific or polyspecific.

The term “chimeric antibody” refers to an antibody in which a portion of the heavy and/or light chain is derived from a particular source or species, while the remainder of the heavy and/or light chain is derived from a different source or species.

An “isolated antibody” or “isolated nucleic acid” is an antibody or nucleic acid that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some aspects, an isolated antibody or isolated nucleic acid is prepared by at least one purification step. In some embodiments, an isolated antibody or isolated nucleic acid is purified to at least 80%, 85%, 90%, 95%, or 99% by weight. In some embodiments, an isolated antibody or isolated nucleic acid is purified to at least 80%, 85%, 90%, 95%, or 99% by volume. In some embodiments, an isolated antibody or isolated nucleic acid is provided as a solution comprising at least 85%, 90%, 95%, 98%, 99% to 100% antibody or nucleic acid by weight. In some embodiments, an isolated antibody or isolated nucleic acid is provided as a solution comprising at least 85%, 90%, 95%, 98%, 99% to 100% RPP or nucleic acid by volume.

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule.

### 6.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

### 6.3. Methods of Analyzing ABPs

The present disclosure provides a method of analyzing a library (“test library”) comprising ABPs.

In some embodiments, the method comprises measuring a distribution of charge state (e.g., pIs (isoelectric points)) of the test library. The method can further comprise comparing the distribution of charge states with a reference distribution, wherein the reference distribution is a distribution of charge states of a reference library. The method can further comprise determining a quality of the test library based on the comparison.

In some embodiments, the method comprises measuring size heterogeneity of the test library. In some embodiments, the method comprises characterizing the high molecular weight species (HMW), polyclonal antibody monomers (pAb peaks), and low molecular weight species (LMW) in the library of ABPs.

In some embodiments, the method disclosed herein is used separately or in combination with other methods. In some embodiments, the method disclosed herein is performed concurrently or sequentially with other analysis method(s).

### 6.3.1. Measurement of Charge States

The method disclosed herein comprises measuring charge states (e.g., pIs) of the test library. Various methods of measuring charge states known in the art can be used. In some embodiments, cation exchange-High Performance Liquid Chromatography (CEX-HPLC) is used to measure charge states. In some embodiments, the HPLC data or profile shows distribution of the charge states. Other methods which separate the antibodies based on the differential charge state (pI values) of component antibodies using a pH gradient can be used in various embodiments disclosed herein.

The CEX-HPLC can be performed with a pH gradient from a mobile phase A to a mobile phase B. In some embodiments, the mobile phase A has a low pH and the mobile phase B has a high pH. In some embodiments, the mobile phase A has a pH from pH 5 to pH 7. In some embodiments, the mobile phase A has a pH from pH 5 to pH 6. In some embodiments, the mobile phase A has a pH from pH 5 to pH 5.5. In some embodiments, the mobile phase A has a pH from pH 5.5 to pH 6. In some embodiments, the mobile phase B has a pH from pH 9 to pH 11. In some embodiments, the mobile phase B has a pH from pH 10 to pH 11. In some embodiments, the mobile phase B has a pH from pH 10 to pH 10.5. In some embodiments, the mobile phase B has a pH from pH 10.5 to pH 11.

In some embodiments, the CEX-HPLC is performed at a flow rate between 0.25 mL/min and 2 mL/min. In some embodiments, the CEX-HPLC is performed at a flow rate between 0.5 mL/min and 2 mL/min. In some embodiments, the CEX-HPLC is performed at a flow rate between 0.5 mL/min and 1.5 mL/min. In some embodiments, the CEX-HPLC is performed at a flow rate between 0.75 mL/min and 1.5 mL/min. In some embodiments, the CEX-HPLC is performed at a flow rate between 0.75 mL/min and 1 mL/min. In some embodiments, the CEX-HPLC is performed at a flow rate between 0.75 mL/min and 0.85 mL/min. In some embodiments, the CEX-HPLC is performed at a flow rate of 0.5 mL/min, 0.75 mL/min or 1.0 mL/min.

In some embodiments, the CEX-HPLC is performed at a temperature between 2° and 40° C. In some embodiments, the CEX-HPLC is performed at a temperature between 25 and 35° C. In some embodiments, the CEX-HPLC is performed at a temperature between 27.5 and 32.5° C. In some embodiments, the CEX-HPLC is performed at 25° C., at 30° C., or at 35° C.

In some embodiments, the CEX-HPLC is performed at a temperature of at least 25° C. In some embodiments, the CEX-HPLC is performed at a temperature of at least 26° C. In some embodiments, the CEX-HPLC is performed at a temperature of at least 27° C. In some embodiments, the CEX-HPLC is performed at a temperature of at least 28° C. In some embodiments, the CEX-HPLC is performed at a temperature of at least 29° C. In some embodiments, the CEX-HPLC is performed at a temperature of at least 30° C. In some embodiments, the CEX-HPLC is performed at a temperature of at least 35° C. In some embodiments, the CEX-HPLC is performed at a temperature of at least 40° C.

In some embodiments, the CEX-HPLC is performed with 1-100% B gradient, 5-100% B gradient, 10-100% B gradient, 15-100% B gradient or 20-100% B gradient.

In some embodiments, the CEX-HPLC is performed with 30-60 min gradient time. In some embodiments, the CEX-HPLC is performed with 30-60 min gradient time, 30-45 min gradient time, or 30-40 min gradient time. In some embodiments, the CEX-HPLC is performed with 35 min gradient time, 40 min gradient time, 45 min gradient time, 50 min gradient time, 55 min gradient time, or 60 min gradient time.

In some embodiments, the CEX-HPLC is performed with a CEX column comprising a weak cation-exchange material. In some embodiments, the CEX-HPLC is performed with a CEX column with a strong cation exchanger. Strong cation exchangers can contain an acid functional group, such as a sulfonic acid, which is ionized over the entire pH range. Weak cation exchange sorbents have surface functional groups such as carboxylic acids that are negatively charged at high pH, but neutral at low pH.

In some embodiments, the method comprises use of a CEX column packed with resin having a particle size smaller than 3 μm. In some embodiments, the particle size is smaller than 2.9 μm, smaller than 2.8 μm, smaller than 2.7 μm, smaller than 2.6 μm, smaller than 2.5 μm, smaller than 2.4 μm, smaller than 2.3 μm, smaller than 2.2 μm, smaller than 2.1 μm, smaller than 2.0 μm, smaller than 1.9 μm, smaller than 1.8 μm, smaller than 1.7 μm, smaller than 1.6 μm, or smaller than 1.5 μm. In some embodiments, the particle size is between 2.3 μm and 2.9 μm, between 2.5 μm and 2.8 μm, between 2.5 μm and 2.8 μm, between 1.5 μm and 2.5 μm, between 1.5 μm and 2.0 μm, between 1.0 μm and 2.0 μm or between 1.0 μm and 1.5 μm.

In some embodiments, the method comprises use of a CEX column packed with a porous resin. In some embodiments, the method comprises use of a CEX column packed with a non-porous resin.

In some embodiments, the CEX column is Proteomix SCX or MabPac SCX-10.

### 6.3.2. Measurement of Size Heterogeneity

In some embodiments, the method disclosed herein comprises measuring size heterogeneity of the test library. Various methods of measuring the size heterogeneity can be used. In some embodiments, SEC-HPLC method which has been improved for high-resolution analysis is used. The high-resolution SEC-HPLC method can characterize the high molecular weight species (HMW), polyclonal antibody monomers (pAb peaks), and low molecular weight species (LMW) in the library of ABPs.

Size Exclusion Chromatography (SEC) separates proteins on the basis of their hydrodynamic radius (size). The column stationary phase includes porous particles through which molecules diffuse using a mobile phase. Small particles are trapped into the pores and elute for a longer time, whereas larger particles do not enter the pores and elute faster than smaller particles. SEC is used to separate a protein monomer from its dimer and high molecular weight (HMW) aggregates, fragments and other low molecular weight (LMW) impurities. In some embodiments, the eluted proteins are monitored by UV absorbance at 280 nm. The area under curve (AUC) quantitatively corresponds to the amount of protein. In some embodiments the percentage of peak area for the monomer, HMW, and LMW are reported.

In some embodiments, the method comprises use of an SEC column packed with resin having a particle size smaller than 3 μm is used. In some embodiments, the particle size is smaller than 2.9 μm, smaller than 2.8 μm, smaller than 2.7 μm, smaller than 2.6 μm, smaller than 2.5 μm, smaller than 2.4 μm or smaller than 2.3 μm. In some embodiments, the particle size is between 2.3 μm and 2.9 μm, between 2.5 μm and 2.8 μm, or between 2.5 μm and 2.8 μm.

In some embodiments, the SEC method uses a stationary phase to reduce residual silanol groups. In some embodiments, the method uses BEH stationary phase. In some embodiments, the materials have an empirical formula of SiO₂ (O_{1.5}SiCH₂CH₂SiO_{1.5})_{0.25}. They can be synthesized by the co-condensation of 1,2-bis(triethoxysilyl) ethane (1 equiv.) with TEOS (4 equiv.).

In some embodiments, the SEC method uses a mobile phase with a pH between pH 6.5 and pH 7.5. In some embodiments, the mobile phase has a pH between pH 6.75 and pH 7.25. In some embodiments, the mobile phase has a pH between pH 6.8 and pH 7.2. In some embodiments, the mobile phase has a pH between pH 6.9 and pH 7.1. In some embodiments, the mobile phase buffer has a pH 7.0.

In some embodiments, the mobile phase comprises a NaCl concentration between 250 mM and 750 mM. In some embodiments, the mobile phase comprises a NaCl concentration between 300 mM and 700 mM. In some embodiments, the mobile phase comprises a NaCl concentration between 400 mM and 600 mM. In some embodiments, the mobile phase comprises a NaCl concentration of 450 mM, 460 mM, 470 mM, 480 mM, 490 mM, 500 mM, 510 mM, 520 mM, 530 mM, 540 mM, or 550 mM.

In some embodiments, the SEC method is performed at the column temperature between 20° C. and 40° C. In some embodiments, the SEC method is performed at the column temperature between 22° C. and 38° C. In some embodiments, the SEC method is performed at the column temperature between 25° C. and 35° C. In some embodiments, the SEC method is performed at the column temperature between 28° C. and 32° C. In some embodiments, the SEC method is performed at the column temperature between 29° C. and 31° C. In some embodiments, the SEC method is performed at the column temperature of 25° C., 26° C., 27° C., 28° C., 29° C., 30° C., 31° C., 32° C., 33° C., 34° C., or 35° C.

### 6.3.3. ABPs and Libraries of ABPs

Each member of the libraries of ABPs (test library or reference library) described herein is a polypeptide that specifically binds an antigen, e.g., an antibody or an antibody fragment. In some embodiments, the antigen is a viral antigen or a bacterial antigen. In some embodiments, the ABPs include cognate pairs of the heavy and light chain CDR sequences. In some embodiments the ABPs are scFvs. In some embodiments the ABPs are full-length antibodies. An ABP may also be any synthetic or genetically engineered protein.

A library of ABPs can comprise at least 100 ABPs, at least 1000 ABPs, or at least 2000 ABPs. In some embodiments, a library of ABPs comprise 100-5000 ABPs. In some embodiments, a library of ABPs comprise 100-2000 ABPs. In some embodiments, a library of ABPs comprise 100-1000 ABPs. In some embodiments, a library of ABPs comprise 1000-3000 ABPs. In some embodiments, a library of ABPs comprise 2000-3000 ABPs.

In some embodiments, the library comprises one or more components other than ABPs. In some embodiments, the library comprises one or more therapeutic agent other than ABPs. In some embodiments, the library is a pharmaceutical composition comprising ABPs and an excipient.

In some embodiments, the test library comprises at least 500 ABPs, at least 1000 ABPs, at least 2000 ABPs, at least 3000 ABPs, at least 4000 ABPs, at least 5000 ABPs, at least 6000 ABPs, at least 7000 ABPs, at least 8000 ABPs, at least 9000 ABPs, or at least 10,000 ABPs.

In some embodiments, the test library comprises less than 50 ABPs, less than 100 ABPs, less than 200 ABPs, less than 300 ABPs, less than 400 ABPs, less than 500 ABPs, less than 1000 ABPs, less than 2000 ABPs less than 3000 ABPs less than 4000 ABPs, less than 5000 ABPs.

In some embodiments, the reference library comprises at least 500 ABPs, at least 1000 ABPs, at least 2000 ABPs, at least 3000 ABPs, at least 4000 ABPs, at least 5000 ABPs, at least 6000 ABPs, at least 7000 ABPs, at least 8000 ABPs, at least 9000 ABPs, or at least 10,000 ABPs.

In some embodiments, the reference library comprises less than 50 ABPs, less than 100 ABPs, less than 200 ABPs, less than 300 ABPs, less than 400 ABPs, less than 500 ABPs, less than 1000 ABPs, less than 2000 ABPs less than 3000 ABPs less than 4000 ABPs, less than 5000 ABPs.

In some embodiments, a plurality of ABPs in a library have binding affinities to the same antigen. In some embodiments, a plurality of ABPs in a library have binding affinities to two or more antigens. In some embodiments, a library comprises ABPs from a single donor. In some embodiments, a library comprises ABPs from multiple donors.

The library can be a test library or a reference library. In some embodiments, a test library and a reference library comprise the same ABPs. In some embodiments, a test library and a reference library comprise different sets of ABPs. In some embodiments, a test library comprises a subset of ABPs in the reference library. In some embodiments, a reference library comprises a subset of ABPs in the test library. In some embodiments, the reference library comprises a subset of the at least 100 ABPs in the test library.

In some embodiments, a reference library comprises one antibody. In some embodiments, a reference library comprises a plurality of antibodies. In some embodiments, the reference library has been generated by mixing a plurality of monoclonal antibodies.

In some embodiments, the library comprises ABPs which have been analyzed by sequencing. In some embodiments, the test library and the reference library are produced from the same production cell line or a progeny thereof. In some embodiments, the reference library has been analyzed by sequencing. In some embodiments, the reference library is a batch different from the test library. In some embodiments, the test library and the reference library have been separately generated.

In some embodiments, the test library has been prepared by a process comprising: generation the at least 100 ABPs by culturing a production cell line; and purification of the at least 100 ABPs. In some embodiments, the purification is performed by at least one of the following steps: affinity chromatography, low pH virus interaction, hydrophobic interaction chromatography or membrane filtration, multimodal anion exchange chromatography or membrane filtration, anion exchange chromatography or membrane filtration, multimodal cation exchange chromatography, cation exchange chromatography, virus filtration, and ultrafiltration and/or diafiltration. In some embodiments, the purification is performed by two, three, four, five, six or all of the following steps: affinity chromatography, low pH virus interaction, hydrophobic interaction chromatography or membrane filtration, multimodal anion exchange chromatography or membrane filtration, anion exchange chromatography or membrane filtration, multimodal cation exchange chromatography, cation exchange chromatography, virus filtration, and ultrafiltration and/or diafiltration.

### 6.3.4. Analysis of Test Library

The method disclosed herein can comprise comparing the distribution of charge states of the test library (i.e., test distribution) with a distribution of charge states of the reference library (i.e., reference distribution). In some embodiments, the comparison is performed by comparing the HPLC profiles of the test library and the reference library.

The comparison can be used to assess the safety, identity, strength, quality and potency of the library. In some embodiments, the analysis is used to assess the physiochemical properties of the test library. A panel of qualified release and additional characterization methods can be used to characterize and control the test library. The analytical methods and acceptance criteria can be developed and adjusted based on information obtained from characterization of process development batches.

The reference distribution can be measured by the method used for measuring the charge states of the test library. In some embodiments, CEX-HPLC is used to measure the reference distribution. In some embodiments, the reference distribution is measured by the method and condition used to measure the charge states of the test library. In some embodiments, the reference distribution is measured by cation exchange-High Performance Liquid Chromatography (CEX-HPLC) under a condition identical to the condition of measuring the distribution of charge states of the test library.

In some embodiments, the reference distribution is obtained by bioinformatic analysis. In some embodiments, the reference distribution is obtained by analyzing amino acid sequences of ABPs. In some embodiments, the reference distribution and average of molecular weight, charge state, and extinction coefficient of the library components are computed using the component amino acid sequences.

In some embodiments, the reference distribution is obtained based on sequence analysis of ABPs. In some embodiments, the reference distribution is obtained based on sequence analysis of ABPs expected to be present in the test library. In some embodiments, the reference distribution is obtained based on sequence analysis of ABPs in the reference library.

In some embodiments, the analysis method comprises comparing charge states of the test library and charge states of the reference library. In some embodiments, the comparison comprises comparing the peak retention time, peak area, peak height and/or the number of peaks. In some embodiments, the comparison is performed by identifying overlapping peaks. In some embodiments, the comparison is performed by comparing the area under curve (AUC) of the HPLC profiles between the test library and the reference library. In some embodiments, the comparison indicates the safety, identity, strength, quality or potency of the library.

In some embodiments, the test library is determined to have a better quality when its distribution of charge states is closer to the reference distribution. In some embodiments, the test library is determined to have a good quality when its distribution of charge states is at least 90%, 96%, 97%, 98%, or 99% identical to the reference distribution in terms of the peak retention time, peak area, peak height and/or the number of peaks. In some embodiments, the test library is determined to have a good quality when its distribution of charge states is at least 90%, 96%, 97%, 98%, or 99% identical to the reference distribution in terms of area under curve (AUC).

In some embodiments, the overlap of the main peaks between the test library and the reference library is compared.

In some embodiments, overlap of charge states between the test library and the reference library correlates with the safety, identity, strength, quality or potency of the library.

In some embodiments, one or more additional factors are considered for determination of the quality or characterization of the test library.

In some embodiments, one or more physical properties of the test library are measured to determine whether they are within acceptance ranges. The physical properties are one or more selected from color, clarity, visible particulates, pH, osmolarity, PS20, protein concentration, antigen binding, size variants, denatured size distribution, bacteria endotoxins, sterility, and sub-visible particulates.

In some embodiments, the test library is acceptable when it is not more intensely colored than a reference material. In some embodiments, the test library is acceptable when it is not more opalescent than a reference material. In some embodiments, the test library is acceptable when it is essentially free of visible particles. In some embodiments, the test library is acceptable when its pH is between pH 3 and pH 6, between pH 4 and pH 5, between pH 4.5 and pH 5 or between pH 4.8 and pH 4.9. In some embodiments, the test library is acceptable when its osmolarity is between 300 and 500 mOsm/kg, between 300 and 400 mOsm/kg, between 325 and 375 mOsm/kg, or between 340 and 360 mOsm/kg. In some embodiments, the test library is acceptable when it includes Polysorbate 20 (PS20) at the concentration of 0.001% to 0.1% w/v, 0.005% to 0.05% w/v, 0.075% to 0.03% w/v, or 0.01% to 0.03% w/v. In some embodiments, the test library is acceptable when its protein concentration is 20 to 40 mg/mL, 25 to 35 mg/mL, or about 30 mg/mL.

In some embodiments, the test library is acceptable when its binding to a target antigen is comparable to a reference material. In some embodiments, the test library is acceptable when its binding to a target antigen is 25%-300%, 50%-200%, or 50%-150% of a reference material. In some embodiments, the binding is measured by ELISA. In some embodiments, the test library is acceptable when its neutralizing activity against a target (e.g. virus) is comparable to a reference material. In some embodiments, the test library is acceptable when its neutralizing activity against a target is 25%-300%, 50%-200%, or 50%-150% of a reference material.

In some embodiments, the test library is acceptable when SEC (size exclusion chromatography) by HPLC indicates that peaks corresponding to polyclonal antibodies represent more than 50%, more than 60%, more than 70%, more than 80%, more than 90%, more than 95%, more than 98% or more than 99% of the entire signals. In some embodiments, the test library is acceptable when SEC by HPLC indicates that peaks corresponding to high molecular weight (HMW) molecules are less than 10%, less than 7.5%, less than 5% or less than 2.5%. In some embodiments, the test library is acceptable when SEC by HPLC indicates that peaks corresponding to low molecular weight (LMW) molecules are less than 10%, less than 7.5%, less than 5% or less than 2.5%.

In some embodiments, the test library is acceptable when denatured size distribution shows that more than 50%, more than 60%, more than 70%, more than 80%, more than 85%, more than 90% or more than 95% of the signals correspond to intact antibodies. In some embodiments, the test library is acceptable when denatured size distribution shows that more than 70%, more than 75%, more than 80%, more than 85%, more than 90% or more than 95% of the signals correspond to a heavy chain or a light chain of an antibody.

In some embodiments, the quality of the test library is determined further based on antigen binding of the test library measured by ELISA. The antigen binding of the test library can be compared to the reference material.

In some embodiments, the quality of the test library is determined further based on analysis of the test library by size exclusion chromatography (SEC)-HPLC. In some embodiments, the quality of the test library is determined further based on amino acid sequences of IgG1 and IgK framework. The amino acid sequences of IgG1 and IgK framework can be determined by an LC-MS reduced peptide mapping method. In some embodiments, the quality of the test library is determined further based on a disulfide bond linkage between IgG1 and IgK constant region. In some embodiments, the disulfide bond linkage between IgG1 and IgK constant region is determined by an LC-MS non-reduced peptide mapping method. In some embodiments, the quality of the test library is determined further based on a size heterogeneity of the test library. In some embodiments, the size heterogeneity of the test library is characterized by a multi-angle light scattering (MALS) technique. In some embodiments, the quality of the test library is determined further based on a melting temperature of the test library. In some embodiments, the melting temperature is measured by differential scanning calorimetry (DSC). In some embodiments, the quality of the test library is determined further based on a glass transition temperature of the test library. In some embodiments, the glass transition temperature of the test library is measured by differential scanning calorimetry (DSC). In some embodiments, the quality of the test library is determined further based on released N-glycan analysis. N-linked glycans of the library can be released by enzymatic cleavage (PNGase F) under reducing and denaturing conditions, the N-glycans can be fluorescently tagged, separated by HILIC-UPLC chromatography, and detected by a mass spectrometer. In some embodiments, the quality of the test library is determined further based on total sialic acid quantification. In some embodiments, sialic acid is released and measured under native conditions using AdvanceBio total sialic acid quantitation kit from Agilent. The kit uses enzymatic cleavage for sialic acid release, followed by conversion of sialic acid to H₂O₂, and detection with a fluorescent reporter dye. In some embodiments, the quality of the test library is determined further based on hemagglutination method.

### 6.4. Methods of Producing and Preparing a Library of ABPs

The analysis method disclosed herein can be used to analyze or characterize a library comprising any of the ABPs disclosed herein. ABPs can be purified from host cells that have been transfected by a gene encoding the antibodies. The purification can be performed by any method known in the art, for example, elution of filtered supernatant of host cell culture fluid using a Heparin HP column, using a salt gradient, or with protein A resin.

ABPs provided herein can be naturally occurring antibodies or engineered antibodies. The variable region domains of ABPs can be any naturally occurring variable domain or an engineered version thereof. By engineered version is meant a variable region domain that has been created using recombinant DNA engineering techniques. Such engineered versions include those created, for example, from a specific antibody variable region by insertions, deletions, or changes in or to the amino acid sequences of the specific antibody. Particular examples include engineered variable region domains containing at least one CDR and optionally one or more framework amino acids from a first antibody and the remainder of the variable region domain from a second antibody.

The variable region domain may be covalently attached at a C terminal amino acid to at least one other antibody domain or a fragment thereof. Thus, for example, a V_{H} domain that is present in the variable region domain may be linked to an immunoglobulin CH1 domain, or a fragment thereof. Similarly, a V_{L} domain may be linked to a CK domain or a fragment thereof. In this way, for example, the antibody may be a Fab fragment wherein the antigen binding domain contains associated V_{H} and V_{L} domains covalently linked at their C termini to a CH1 and CK domain, respectively. The CH1 domain may be extended with further amino acids, for example to provide a hinge region or a portion of a hinge region domain as found in a Fab′ fragment, or to provide further domains, such as antibody CH2 and CH3 domains.

Fully human monoclonal antibodies may be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B-cells, fusion of spleen cells from immunized transgenic mice carrying inserted human immunoglobulin genes, isolation from human immunoglobulin V region phage libraries, or other procedures as known in the art and based on the disclosure herein. For example, fully human monoclonal antibodies may be obtained from transgenic mice that have been engineered to produce specific human antibodies in response to antigenic challenge. Methods for obtaining fully human antibodies from transgenic mice are described, for example, by Green et al., Nature Genet. 7:13, 1994; Lonberg et al., Nature 368:856, 1994; Taylor et al., Int. Immun. 6:579, 1994; U.S. Pat. No. 5,877,397; Bruggemann et al., 1997 Curr. Opin. Biotechnol. 8:455-58; Jakobovits et al., 1995 Ann. N. Y. Acad. Sci. 764:525-35. In this technique, elements of the human heavy and light chain locus are introduced into strains of mice derived from embryonic stem cell lines that contain targeted disruptions of the endogenous heavy chain and light chain loci (see also Bruggemann et al., Curr. Opin. Biotechnol. 8:455-58 (1997)). For example, human immunoglobulin transgenes may be mini-gene constructs, or transloci on yeast artificial chromosomes, which undergo B-cell-specific DNA rearrangement and hypermutation in the mouse lymphoid tissue. Fully human monoclonal antibodies may be obtained by immunizing the transgenic mice, which may then produce human antibodies specific for the antigen target or targets. Lymphoid cells of the immunized transgenic mice can be used to produce human antibody-secreting hybridomas according to the methods described herein.

In some embodiments, ABPs comprise the cognate pairs of heavy and light chain CDR3 sequence disclosed herein. For example, CDRs may be incorporated into known antibody framework regions (IgG1, IgG2, etc.), or conjugated to a suitable vehicle to enhance the half-life thereof. Suitable vehicles include, but are not limited to Fc, polyethylene glycol (PEG), albumin, transferrin, and the like. These and other suitable vehicles are known in the art. Such conjugated CDR peptides may be in monomeric, dimeric, tetrameric, or other form. In one embodiment, one or more water-soluble polymer is bonded at one or more specific position, for example at the amino terminus, of a binding agent.

Antigen-binding fragments of ABPs of the invention can be produced by conventional techniques. Examples of such fragments include, but are not limited to, Fab and F(ab′)₂ fragments. Antibody fragments and derivatives produced by genetic engineering techniques also are contemplated.

Additional embodiments include chimeric antibodies, e.g., humanized versions of non-human (e.g., murine) monoclonal antibodies. Such humanized antibodies may be prepared by known techniques, and offer the advantage of reduced immunogenicity when the antibodies are administered to humans. In one embodiment, a humanized antibody comprises the variable domain of a murine antibody (or all or part of the antigen binding site thereof) and a constant domain derived from a human antibody. Alternatively, a humanized antibody fragment may comprise the antigen binding site of a murine antibody and a variable domain fragment (lacking the antigen-binding site) derived from a human antibody. Procedures for the production of chimeric and further engineered antibodies include those described in Riechmann et al., 1988, Nature 332:323, Liu et al., 1987, Proc. Nat. Acad. Sci. USA 84:3439, Larrick et al., 1989, Bio/Technology 7:934, and Winter et al., 1993, TIPS 14:139. In one embodiment, the chimeric antibody is a CDR grafted antibody. Techniques for humanizing antibodies are discussed in, e.g., U.S. Pat. Nos. 5,869,619, 5,225,539, 5,821,337, 5,859,205, 6,881,557, Padlan et al., 1995, FASEB J. 9:133-39, and Tamura et al., 2000, J. Immunol. 164:1432-41.

Another method for generating human antibodies of the invention includes immortalizing human peripheral blood cells by EBV transformation. See, e.g., U.S. Pat. No. 4,464,456. Such an immortalized B-cell line (or lymphoblastoid cell line) producing an ABP that specifically binds to target or targets can be identified by immunodetection methods as provided herein, for example, an ELISA, and then isolated by standard cloning techniques. The stability of the lymphoblastoid cell line producing an ABP may be improved by fusing the transformed cell lines with a murine myeloma to produce a mouse-human hybrid cell line according to methods known in the art (see, e.g., Glasky et al., Hybridoma 8:377-89 (1989)). Still another method to generate human ABPs is in vitro immunization, which includes priming human splenic B-cells with antigen targets, followed by fusion of primed with a heterohybrid fusion partner. See, e.g., Boerner et al., 1991 J. Immunol. 147:86-95.

In certain embodiments, B-cells that are producing an ABP are selected and the light chain and heavy chain variable regions are cloned from the B-cell according to molecular biology techniques known in the art (WO 92/02551; U.S. Pat. No. 5,627,052; Babcook et al., Proc. Natl. Acad. Sci. USA 93:7843-48 (1996)) and described herein. B-cells from an immunized animal may be isolated from the spleen, lymph node, or peripheral blood sample by selecting a cell that is producing an antibody that specifically binds to to the antigen target. B-cells may also be isolated from humans, for example, from a peripheral blood sample.

Methods for detecting single B-cells that are producing an antibody with the desired specificity are well known in the art, for example, by plaque formation, fluorescence-activated cell sorting, in vitro stimulation followed by detection of specific antibody, and the like. Methods for selection of specific antibody-producing B-cells include, for example, preparing a single cell suspension of B-cells in soft agar that contains the antigen target. Binding of the specific antibodies produced by the B-cell to the antigen results in the formation of a complex, which may be visible as an immunoprecipitate.

In some embodiments, specific antibody-producing B-cells are selected by using a method that allows identification natively paired antibodies. For example, a method described in Adler et al., A natively paired antibody library yields drug leads with higher sensitivity and specificity than a randomly paired antibody library, (Adler et al., Mabs 9, 1282-1996, 2017), which is incorporated by reference in its entirety herein, can be employed. The method combines microfluidic technology, molecular genomics, yeast single-chain variable fragment (scFv) display, fluorescence-activated cell sorting (FACS) and deep sequencing. In short, B cells can be isolated from immunized animals and then pooled. The B cells are encapsulated into droplets with oligo-dT beads and a lysis solution, and mRNA-bound beads are purified from the droplets, and then injected into a second emulsion with an OE-RT-PCR amplification mix that generates DNA amplicons that encode scFv with native pairing of heavy and light chain Ig. Libraries of natively paired amplicons are then electroporated into yeast for scFv display. FACS is used to identify high affinity scFv. Finally, deep antibody sequencing can be used to identify all clones in the pre- and post-sort scFv libraries.

After the B-cells producing the desired antibody are selected, the specific antibody genes may be cloned by isolating and amplifying DNA or mRNA according to methods known in the art and described herein.

In some embodiments, ABPs are generated by the method described in Example 1-5 of the present disclosure. In some embodiments, the ABPs are those generated and disclosed in PCT/US2020/030878 filed on Apr. 30, 2020; PCT/US2021/037232 filed on Jun. 14, 2021; and PCT/US2021/044523 filed on Aug. 4, 2021, which are incorporated by reference in their entireties.

The methods for obtaining antibodies of the invention can also adopt various phage display technologies known in the art. See, e.g., Winter et al., 1994 Annu. Rev. Immunol. 12:433-55; Burton et al., 1994 Adv. Immunol. 57:191-280. Human or murine immunoglobulin variable region gene combinatorial libraries may be created in phage vectors that can be screened to select Ig fragments (Fab, Fv, sFv, or multimers thereof) that bind specifically to the ABP or variant or fragment thereof. See, e.g., U.S. Pat. No. 5,223,409; Huse et al., 1989 Science 246:1275-81; Sastry et al., Proc. Natl. Acad. Sci. USA 86:5728-32 (1989); Alting-Mees et al., Strategies in Molecular Biology 3:1-9 (1990); Kang et al., 1991 Proc. Natl. Acad. Sci. USA 88:4363-66; Hoogenboom et al., 1992 J. Molec. Biol. 227:381-388; Schlebusch et al., 1997 Hybridoma 16:47-52 and references cited therein. For example, a library containing a plurality of polynucleotide sequences encoding Ig variable region fragments may be inserted into the genome of a filamentous bacteriophage, such as M13 or a variant thereof, in frame with the sequence encoding a phage coat protein. A fusion protein may be a fusion of the coat protein with the light chain variable region domain and/or with the heavy chain variable region domain. According to certain embodiments, immunoglobulin Fab fragments may also be displayed on a phage particle (see, e.g., U.S. Pat. No. 5,698,426).

In one embodiment, in a hybridoma the variable regions of a gene expressing a monoclonal antibody of interest are amplified using nucleotide primers. These primers may be synthesized by one of ordinary skill in the art or may be purchased from commercially available sources. (See, e.g., Stratagene (La Jolla, California), which sells primers for mouse and human variable regions including, among others, primers for V_{Ha}, V_{Hb}, V_{Hc}, V_{Hd}, C_{H1}, V_{L} and C_{L} regions.) These primers may be used to amplify heavy or light chain variable regions, which may then be inserted into vectors such as ImmunoZAP™ H or ImmunoZAP™ L (Stratagene), respectively. These vectors may then be introduced into E. coli, yeast, or mammalian-based systems for expression. Large amounts of a single-chain protein containing a fusion of the V_{H} and V_{L} domains may be produced using these methods (see Bird et al., Science 242:423-426, 1988).

ABPs (e.g., antibodies, antibody fragments, and antibody derivatives) of the invention can comprise any constant region known in the art. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. In one embodiment, the light or heavy chain constant region is a fragment, derivative, variant, or mutein of a naturally occurring constant region.

Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype. See also Lantto et al., 2002, Methods Mol. Biol. 178:303-16.

Single chain antibodies (scFv) may be formed by linking heavy and light chain variable domain (Fv region) fragments via an amino acid bridge (short peptide linker, e.g., a synthetic sequence of amino acid residues), resulting in a single polypeptide chain. Such single-chain Fvs (scFvs) have been prepared by fusing DNA encoding a peptide linker between DNAs encoding the two variable domain polypeptides (V_{L} and V_{H}). The resulting polypeptides can fold back on themselves to form antigen-binding monomers, or they can form multimers (e.g., dimers, trimers, or tetramers), depending on the length of a flexible linker between the two variable domains (Kortt et al., 1997, Prot. Eng. 10:423; Kortt et al., 2001, Biomol. Eng. 18:95-108, Bird et al., 1988, Science 242:423-26 and Huston et al., 1988, Proc. Natl. Acad. Sci. USA 85:5879-83). By combining different V_{L} and V_{H}-comprising polypeptides, one can form multimeric scFvs that bind to different epitopes (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, de Graaf et al., 2002, Methods Mol Biol. 178:379-87.

In certain aspects, the invention includes ABPs generated from libraries of antibody-encoding expression vectors. The library comprises 10, 100, 1,000, 10,000 or more than 100,000 distinct antibody sequences. In certain aspects, the ABPs are generated from mammalian cells engineered recombinantly with antibody sequences encoded by single plasma cells or plasmablasts. In certain aspects, the ABPs are polyvalent, in that they comprise antibodies that have different antigen-binding properties. In some embodiments, the ABPs bind to multiple epitopes on a target antigen. In some embodiments, the ABPs bind to multiple antigens.

Once cells producing antibodies according to the invention have been obtained using any of techniques known in the art, the specific antibody genes may be cloned by isolating and amplifying DNA or mRNA therefrom according to standard procedures as described herein. The antibodies produced therefrom may be sequenced and the CDRs identified and the DNA coding for the CDRs may be manipulated as described previously to generate other antibodies according to the invention.

ABPs of the present invention preferably have activity in the cell-based methods described herein and/or the in vivo method described herein and/or bind to one or more of the domains described herein. Accordingly, such binding agents can be identified using the methods described herein.

Other antibodies according to the invention may be obtained by conventional immunization and cell fusion procedures as described herein and known in the art.

Molecular evolution of the complementarity determining regions (CDRs) in the center of the antibody binding site also has been used to isolate antibodies with increased affinity, for example, antibodies having increased affinity for c-erbB-2, as described by Schier et al., 1996, J. Mol. Biol. 263:551.

Although human, partially human, or humanized antibodies will be suitable for many applications, particularly those involving administration of the antibody to a human subject, other types of antigen binding proteins will be suitable for certain applications. The non-human antibodies of the invention can be, for example, derived from any antibody-producing animal, such as mouse, rat, rabbit, goat, donkey, or non-human primate (such as monkey (e.g., cynomologous or rhesus monkey) or ape (e.g., chimpanzee)). Non-human antibodies of the invention can be used, for example, in in vitro and cell-culture based applications, or any other application where an immune response to the antibody of the invention does not occur, is insignificant, can be prevented, is not a concern, or is desired. In one embodiment, a non-human antibody of the invention is administered to a non-human subject. In another embodiment, the non-human antibody does not elicit an immune response in the non-human subject. In another embodiment, the non-human antibody is from the same species as the non-human subject, e.g., a mouse antibody of the invention is administered to a mouse. An antibody from a particular species can be made by, for example, immunizing an animal of that species with the desired immunogen or using an artificial system for generating antibodies of that species (e.g., a bacterial or phage display-based system for generating antibodies of a particular species), or by converting an antibody from one species into an antibody from another species by replacing, e.g., the constant region of the antibody with a constant region from the other species, or by replacing one or more amino acid residues of the antibody so that it more closely resembles the sequence of an antibody from the other species. In one embodiment, the antibody is a chimeric antibody comprising amino acid sequences derived from antibodies from two or more different species.

Antigen binding proteins may be prepared, and screened for desired properties, by any of a number of conventional techniques. Certain of the techniques involve isolating a nucleic acid encoding a polypeptide chain (or portion thereof) of an ABP of interest, and manipulating the nucleic acid through recombinant DNA technology. The nucleic acid may be fused to another nucleic acid of interest, or altered (e.g., by mutagenesis or other conventional techniques) to add, delete, or substitute one or more amino acid residues, for example. Furthermore, the antigen binding proteins may be purified from cells that naturally express them (e.g., an antibody can be purified from a hybridoma that produces it), or produced in recombinant expression systems, using any technique known in the art. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, NY, (1988).

Any expression system known in the art can be used to make the recombinant polypeptides of the invention. Expression systems are detailed comprehensively above. In general, host cells are transformed with a recombinant expression vector that comprises DNA encoding a desired polypeptide. Among the host cells that may be employed are prokaryotes, yeast or higher eukaryotic cells. Prokaryotes include gram negative or gram-positive organisms, for example E. coli or Bacilli. Higher eukaryotic cells include insect cells and established cell lines of mammalian origin. Examples of suitable mammalian host cell lines include the COS-7 line of monkey kidney cells (ATCC CRL 1651) (Gluzman et al., 1981, Cell 23:175), L cells, 293 cells, C127 cells, 3T3 cells (ATCC CCL 163), Chinese hamster ovary (CHO) cells, HeLa cells, BHK (ATCC CRL 10) cell lines, and the CVI/EBNA cell line derived from the African green monkey kidney cell line CVI (ATCC CCL 70) as described by McMahan et al., 1991, EMBO J. 10:2821. Appropriate cloning and expression vectors for use with bacterial, fungal, yeast, and mammalian cellular hosts are described by Pouwels et al. (Cloning Vectors: A Laboratory Manual, Elsevier, New York, 1985).

Production cell lines for monoclonal antibodies (mAbs) are typically produced by randomly inserting expression constructs into a mammalian production cell genome, for example, a CHO genome (Rita Costa et al., 2010). However, this canonical method produces cell lines with multiple copies of mAb inserted into the CHO genome. If we randomly inserted our polyclonal antibody construct libraries into the CHO genome, many clones would express multiple antibodies, which would result in frequent non-native pairing between heavy and light chain Ig. Additionally, different genome locations have different transcriptional activity levels (Kito et al., 2002), which could result in heterogeneous, inconsistent and/or unstable bioproduction. Thus, in some aspects the current invention provides a CHO cell line with a Flp recombinase recognition target (FRT) landing pad stably engineered into the genome. Such site-directed genome integration cell lines can be used for stable expression of ABPs.

It will be appreciated that an antibody of the present invention may have at least one amino acid substitution, providing that the antibody retains binding specificity. Therefore, modifications to the antibody structures are encompassed within the scope of the invention. These may include amino acid substitutions, which may be conservative or non-conservative that do not destroy the binding capability of an antibody comprising the ABP. Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties. A conservative amino acid substitution may also involve a substitution of a native amino acid residue with a normative residue such that there is little or no effect on the polarity or charge of the amino acid residue at that position.

Non-conservative substitutions may involve the exchange of a member of one class of amino acids or amino acid mimetics for a member from another class with different physical properties (e.g. size, polarity, hydrophobicity, charge). Such substituted residues may be introduced into regions of the human antibody that are homologous with non-human antibodies, or into the non-homologous regions of the molecule.

Moreover, one skilled in the art may generate test variants containing a single amino acid substitution at each desired amino acid residue. The variants can then be screened using activity methods known to those skilled in the art. Such variants could be used to gather information about suitable variants. For example, if one discovered that a change to a particular amino acid residue resulted in destroyed, undesirably reduced, or unsuitable activity, variants with such a change may be avoided. In other words, based on information gathered from such routine experiments, one skilled in the art can readily determine the amino acids where further substitutions should be avoided either alone or in combination with other mutations.

A skilled artisan will be able to determine suitable variants of the polypeptide as set forth herein using well-known techniques. In certain embodiments, one skilled in the art may identify suitable areas of the molecule that may be changed without destroying activity by targeting regions not believed to be important for activity. In certain embodiments, one can identify residues and portions of the molecules that are conserved among similar polypeptides. In certain embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues which are important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of an antibody with respect to its three-dimensional structure. In certain embodiments, one skilled in the art may choose not to make radical changes to amino acid residues predicted to be on the surface of the protein, since such residues may be involved in important interactions with other molecules.

A number of scientific publications have been devoted to the prediction of secondary structure. See Moult J., Curr. Op. in Biotech., 7 (4): 422-427 (1996), Chou et al., Biochem., 13 (2): 222-245 (1974); Chou et al., Biochem., 113 (2): 211-222 (1974); Chou et al., Adv. Enzymol. Relat. Areas Mol. Biol., 47:45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural database (PDB) has provided enhanced predictability of secondary structure, including the potential number of folds within a polypeptide's or protein's structure. See Holm et al., Nucl. Acid. Res., 27 (1): 244-247 (1999). It has been suggested (Brenner et al., Curr. Op. Struct. Biol., 7 (3): 369-376 (1997)) that there are a limited number of folds in a given polypeptide or protein and that once a critical number of structures have been resolved, structural prediction will become dramatically more accurate.

Additional methods of predicting secondary structure include “threading” (Jones, D., Curr. Opin. Struct. Biol., 7 (3): 377-87 (1997); Sippl et al., Structure, 4 (1): 15-19 (1996)), “profile analysis” (Bowie et al., Science, 253:164-170 (1991); Gribskov et al., Meth. Enzym., 183:146-159 (1990); Gribskov et al., Proc. Nat. Acad. Sci., 84 (13): 4355-4358 (1987)), and “evolutionary linkage” (See Holm, supra (1999), and Brenner, supra (1997)).

In certain embodiments, variants of antibodies include glycosylation variants wherein the number and/or type of glycosylation site has been altered compared to the amino acid sequences of a parent polypeptide. In certain embodiments, variants comprise a greater or a lesser number of N-linked glycosylation sites than the native protein. An N-linked glycosylation site is characterized by the sequence: Asn-X-Ser or Asn-X-Thr, wherein the amino acid residue designated as X can be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred antibody variants include cysteine variants wherein one or more cysteine residues are deleted from or substituted for another amino acid (e.g., serine) as compared to the parent amino acid sequence. Cysteine variants can be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines.

According to certain embodiments, preferred amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physiochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

In certain embodiments, antibodies of the invention may be chemically bonded with polymers, lipids, or other moieties.

The binding agents may comprise at least one of the CDRs described herein incorporated into a biocompatible framework structure. In one example, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions or substitutions of amino acids, relative to a naturally occurring polypeptide or fold. These scaffolds can be derived from a polypeptide of any species (or of more than one species), such as a human, other mammal, other vertebrate, invertebrate, plant, bacteria or virus.

Typically, the biocompatible framework structures are based on protein scaffolds or skeletons other than immunoglobulin domains. For example, those based on fibronectin, ankyrin, lipocalin, neocarzinostain, cytochrome b, CP1 zinc finger, PST1, coiled coil, LACI-D1, Z domain and tendamistat domains may be used (See e.g., Nygren and Uhlen, 1997, Curr. Opin. in Struct. Biol., 7, 463-469).

It will be appreciated that the antibodies of the invention include the humanized antibodies described herein. Humanized antibodies such as those described herein can be produced using techniques known to those skilled in the art (Zhang, W., et al., Molecular Immunology. 42 (12): 1445-1451, 2005; Hwang W. et al., Methods. 36 (1): 35-42, 2005; Dall'Acqua W F, et al., Methods 36 (1): 43-60, 2005; and Clark, M., Immunology Today. 21 (8): 397-402, 2000).

The polypeptides and proteins of the present invention can be purified according to protein purification techniques well known to those of skill in the art. These techniques involve, at one level, the crude fractionation of the proteinaceous and non-proteinaceous fractions. Having separated the peptide polypeptides from other proteins, the peptide or polypeptide of interest can be further purified using chromatographic and electrophoretic techniques to achieve partial or complete purification (or purification to homogeneity). The term “purified polypeptide” as used herein, is intended to refer to a composition, isolatable from other components, wherein the polypeptide is purified to any degree relative to its naturally-obtainable state. A purified polypeptide therefore also refers to a polypeptide that is free from the environment in which it may naturally occur. Generally, “purified” will refer to a polypeptide composition that has been subjected to fractionation to remove various other components, and which composition substantially retains its expressed biological activity. Where the term “substantially purified” is used, this designation will refer to a peptide or polypeptide composition in which the polypeptide or peptide forms the major component of the composition, such as constituting about 50%, about 60%, about 70%, about 80%, about 85%, or about 90% or more of the proteins in the composition.

Various techniques suitable for use in purification will be well known to those of skill in the art. These include, for example, precipitation with ammonium sulphate, PEG, antibodies (immunoprecipitation) and the like or by heat denaturation, followed by centrifugation; chromatography such as affinity chromatography (Protein-A columns), ion exchange, gel filtration, reverse phase, hydroxylapatite, hydrophobic interaction chromatography, isoelectric focusing, gel electrophoresis, and combinations of these techniques. As is generally known in the art, it is believed that the order of conducting the various purification steps may be changed, or that certain steps may be omitted, and still result in a suitable method for the preparation of a substantially purified polypeptide. Exemplary purification steps are provided in the Examples below.

In some embodiments, the purification process comprises one or more steps selected from: Clarification, Affinity Chromatography, Viral Inactivation and Depth Filtration, Hydrophobic Interaction Chromatography or Membrane Filtration, Multimodal Anion Exchange Chromatography or Membrane Filtration, Anion Exchange Chromatography or Membrane Filtration, Multimodal Cation Exchange Chromatography, Cation Exchange Chromatography, Virus Filtration and UF/DF. In some embodiments, protein concentration is measured between each of the steps.

Various methods for quantifying the degree of purification of polypeptide will be known to those of skill in the art in light of the present disclosure. These include, for example, determining the specific binding activity of an active fraction, or assessing the amount of peptide or polypeptide within a fraction by SDS/PAGE analysis. A preferred method for assessing the purity of a polypeptide fraction is to calculate the binding activity of the fraction, to compare it to the binding activity of the initial extract, and to thus calculate the degree of purification, herein assessed by a “-fold purification number.” The actual units used to represent the amount of binding activity will, of course, be dependent upon the particular method technique chosen to follow the purification and whether or not the polypeptide or peptide exhibits a detectable binding activity.

ABPs, e.g., antibodies according to the invention may have a binding affinity for antigen target of less than or equal to 5×10⁻⁷M, less than or equal to 1×10⁻⁷M, less than or equal to 0.5×10⁻⁷M, less than or equal to 1×10⁻⁸ M, less than or equal to 1×10⁻⁹M, less than or equal to 1×10⁻¹⁰ M, less than or equal to 1×10⁻¹¹ M, or less than or equal to 1×10⁻¹² M.

The affinity of an ABP, as well as the extent to which an antibody inhibits binding, can be determined by one of ordinary skills in the art using conventional techniques, for example those described by Scatchard et al. (Ann. N.Y. Acad. Sci. 51:660-672 (1949)) or by surface plasmon resonance (SPR; BIAcore, Biosensor, Piscataway, NJ). For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al., Cancer Res. 53:2560-65 (1993)).

### 6.5. Pharmaceutical Compositions

One aspect of the present disclosure provides a pharmaceutical composition comprising a library of ABPs tested by the method disclosed herein and/or acceptable under the acceptance criteria disclosed herein. In some embodiments, the pharmaceutical composition comprises the test library disclosed above. In some embodiments, the pharmaceutical composition is tested by the method disclosed herein.

Such pharmaceutical compositions comprise a therapeutically or prophylactically effective amount of the ABPs with pharmaceutically acceptable materials, and physiologically acceptable formulation materials. In some embodiments, the effective amount is determined based on the results obtained from the method provided herein.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition.

Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. Neutral buffered saline or saline mixed with conspecific serum albumin are examples of appropriate diluents. In accordance with appropriate industry standards, preservatives such as benzyl alcohol may also be added. The composition may be formulated as a lyophilizate using appropriate excipient solutions (e.g., sucrose) as diluents. Suitable components are nontoxic to recipients at the dosages and concentrations employed. Further examples of components that may be employed in pharmaceutical formulations are presented in Remington's Pharmaceutical Sciences, 16^{th} Ed. (1980) and 20^{th} Ed. (2000), Mack Publishing Company, Easton, PA.

Optionally, the composition additionally comprises one or more physiologically active agents, for example, an anti-angiogenic substance, a chemotherapeutic substance (such as capecitabine, 5-fluorouracil, or doxorubicin), an analgesic substance, etc., non-exclusive examples of which are provided herein. In various particular embodiments, the composition comprises one, two, three, four, five, or six physiologically active agents in addition to an ABP.

In another embodiment of the invention, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any or all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

The optimal pharmaceutical composition will be determined by one skilled in the art depending upon, for example, the intended route of administration, delivery format, and desired dosage. See for example, Remington's Pharmaceutical Sciences, supra. Such compositions may influence the physical state, stability, rate of in vivo release, and rate of in vivo clearance of the polypeptide. For example, suitable compositions may be water for injection, physiological saline solution for parenteral administration.

### 6.5.1. Content of Pharmaceutically Active Ingredient

In some embodiments, the active ingredient (i.e., the proteins and polypeptides of the present invention) is present in the pharmaceutical composition at a concentration of at least 0.01 mg/ml, at least 0.1 mg/ml, at least 0.5 mg/ml, or at least 1 mg/ml. In some embodiments, the pharmaceutical composition comprises at least 10 mg/ml, at least 100 mg/ml, at least 500 m g/ml or at least 1000 mg/ml of ABPs. In some embodiments, the pharmaceutical composition comprises 10 mg/ml to 1000 mg/ml of ABPs. In some embodiments, the pharmaceutical composition comprises 10 mg/ml to 100 mg/ml of ABPs or 10 mg/ml to 500 mg/ml of ABPs.

In certain embodiments, the active ingredient is present in the pharmaceutical composition at a concentration of at least 1 mg/ml, 2 mg/ml, 3 mg/ml, 4 mg/ml, 5 mg/ml, 10 mg/ml, 15 mg/ml, 20 mg/ml, 25 mg/ml, 50 mg/ml, 100 mg/ml, 200 mg/ml, 300 mg/ml, or 500 mg/ml. In certain embodiments, the active ingredient is present in the pharmaceutical composition at a concentration of at least 30 mg/ml, 35 mg/ml, 40 mg/ml, 45 mg/ml or 50 mg/ml.

### 6.5.2. Formulation Generally

The pharmaceutical composition can be in any form appropriate for human or veterinary medicine, including a liquid, an oil, an emulsion, a gel, a colloid, an aerosol or a solid.

The pharmaceutical composition can be formulated for administration by any route of administration appropriate for human or veterinary medicine, including enteral and parenteral routes of administration.

In various embodiments, the pharmaceutical composition is formulated for administration by inhalation. In certain of these embodiments, the pharmaceutical composition is formulated for administration by a vaporizer. In certain of these embodiments, the pharmaceutical composition is formulated for administration by a nebulizer. In certain of these embodiments, the pharmaceutical composition is formulated for administration by an aerosolizer.

In various embodiments, the pharmaceutical composition is formulated for oral administration, for buccal administration, or for sublingual administration.

In some embodiments, the pharmaceutical composition is formulated for intravenous, intramuscular, or subcutaneous administration.

In some embodiments, the pharmaceutical composition is formulated for intrathecal or intracerebroventricular administration.

In some embodiments, the pharmaceutical composition is formulated for topical administration.

In some embodiments, the pharmaceutical composition is formulated for injection or infusion.

### 6.5.3. Pharmacological Compositions Adapted for Injection

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

In various embodiments, the unit dosage form is a vial, ampule, bottle, or pre-filled syringe. In some embodiments, the unit dosage form contains 0.01 mg, 0.1 mg, 0.5 mg, 1 mg, 2.5 mg, 5 mg, 10 mg, 12.5 mg, 25 mg, 50 mg, 75 mg, or 100 mg of the pharmaceutical composition. In some embodiments, the unit dosage form contains 125 mg, 150 mg, 175 mg, or 200 mg of the pharmaceutical composition. In some embodiments, the unit dosage form contains 250 mg of the pharmaceutical composition.

In typical embodiments, the pharmaceutical composition in the unit dosage form is in liquid form. In various embodiments, the unit dosage form contains between 0.1 mL and 50 ml of the pharmaceutical composition. In some embodiments, the unit dosage form contains 1 ml, 2.5 ml, 5 ml, 7.5 ml, 10 ml, 25 ml, or 50 ml of pharmaceutical composition. In some embodiments, the unit dosage form contains more than 50 ml of the pharmaceutical composition.

In particular embodiments, the unit dosage form is a vial containing 1 ml of the pharmaceutical composition at a concentration of 0.01 mg/ml, 0.1 mg/ml, 0.5 mg/ml, or 1 mg/ml. In some embodiments, the unit dosage form is a vial containing 2 ml of the pharmaceutical composition at a concentration of 0.01 mg/ml, 0.1 mg/ml, 0.5 mg/ml, or 1 mg/ml.

In some embodiments, the pharmaceutical composition in the unit dosage form is in solid form, such as a lyophilate, suitable for solubilization.

Unit dosage form embodiments suitable for subcutaneous, intradermal, or intramuscular administration include preloaded syringes, auto-injectors, and autoinject pens, each containing a predetermined amount of the pharmaceutical composition described hereinabove.

In various embodiments, the unit dosage form is a preloaded syringe, comprising a syringe and a predetermined amount of the pharmaceutical composition. In certain preloaded syringe embodiments, the syringe is adapted for subcutaneous administration. In certain embodiments, the syringe is suitable for self-administration. In particular embodiments, the preloaded syringe is a single use syringe.

In various embodiments, the preloaded syringe contains about 0.1 mL to about 0.5 mL of the pharmaceutical composition. In certain embodiments, the syringe contains about 0.5 mL of the pharmaceutical composition. In specific embodiments, the syringe contains about 1.0 mL of the pharmaceutical composition. In particular embodiments, the syringe contains about 2.0 mL of the pharmaceutical composition.

In certain embodiments, the unit dosage form is an autoinject pen. The autoinject pen comprises an autoinject pen containing a pharmaceutical composition as described herein. In some embodiments, the autoinject pen delivers a predetermined volume of pharmaceutical composition. In other embodiments, the autoinject pen is configured to deliver a volume of pharmaceutical composition set by the user.

In various embodiments, the autoinject pen contains about 0.1 mL to about 5.0 mL of the pharmaceutical composition. In specific embodiments, the autoinject pen contains about 0.5 mL of the pharmaceutical composition. In particular embodiments, the autoinject pen contains about 1.0 mL of the pharmaceutical composition. In other embodiments, the autoinject pen contains about 5.0 mL of the pharmaceutical composition.

### 6.5.4. Mixtures of Plasma IVIg with Recombinant Hyperimmunes

In some embodiments, a recombinant hyperimmune is spiked into conventional plasma IVIg to increase the anti-pathogen titer of IVIg. In some embodiments, several anti-pathogen recombinant hyperimmunes are spiked into conventional plasma IVIg, for example, hyperimmunes directed against Hib, pneumococcus, influenza A virus, and tetanus are concurrently spiked into plasma IVIg to treat patients with primary immune deficiency. The spike in hyperimmunes increases the titer of antibodies directed against pathogens to which primary immune deficiency patients are particularly susceptible. Any number of spike-ins can be mixed with plasma IVIg to generate increased titers against any number of pathogens.

In some embodiments, the spike-in recombinant hyperimmunes are mixed with plasma IVIg by the pharmacist. In some embodiments, the spike-in recombinant hyperimmunes are mixed with plasma IVIg by the manufacturer.

### 6.6. Methods of Use

The method disclosed herein can be used for analyzing or characterizing a library comprising ABPs during or after production of the library. The method disclosed herein can be used for analyzing or characterizing a cell line for production of the library comprising ABPs. In some embodiments, the method is used to select a cell line that can produce a high-quality library comprising ABPs. In some embodiments, the method is used to confirm production of the ABPs and/or ensure the quality of the ABPs. In some embodiments, the method is used before, during or after production of a pharmaceutical composition comprising the library of ABPs. In some embodiments, the method is used to test the pharmaceutical composition during or after distribution. In some embodiments, the method is used to test the pharmaceutical composition before therapeutic use of the composition. Accordingly, the present disclosure provides a method of treating a patient with a library comprising ABPs previously tested by the method disclosed herein.

The terms “treatment,” “treating,” and the like are used herein to generally mean obtaining a desired pharmacologic and/or physiologic effect. The effect may be prophylactic, in terms of completely or partially preventing a disease, condition, or symptoms thereof, and/or may be therapeutic in terms of a partial or complete cure for a disease or condition and/or adverse effect, such as a symptom, attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms). Improvements in any conditions can be readily assessed according to standard methods and techniques known in the art. The population of subjects treated by the method of the disease includes subjects suffering from the undesirable condition or disease, as well as subjects at risk for development of the condition or disease.

In vivo and/or in vitro methods may optionally be employed to help identify optimal dosage ranges. The precise dose to be employed in the formulation will also depend on the route of administration, and the seriousness of the condition, and should be decided according to the judgment of the practitioner and each subject's circumstances. Effective doses may be extrapolated from dose-response curves derived from in vitro or animal model test systems.

In some embodiments, the method disclosed herein is used to ensure the quality or potency of the ABPs in the library. In some embodiments, the method disclosed herein is used to adjust optimal dose ranges for therapeutic use of the library.

The actual amount administered, and rate and time-course of administration, will depend on the nature and severity of disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

In some embodiments, the pharmaceutical composition is administered by inhalation, orally, by buccal administration, by sublingual administration, by injection or by topical application.

In some embodiments, the pharmaceutical composition is administered once a day, 2-4 times a day, 2-4 times a week, once a week, or once every two weeks.

### 7. EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### 7.1.1. Example 1: Generation of a Library of ABPs with Activity Against Human Thymocytes or T Cells

Four libraries of ABPs targeting human thymocytes or T cells, i.e., recombinant human anti-thymocyte globulin (rhATG) were produced. Both in vitro and in vivo studies were used to demonstrate functional similarity between this rhATG and the commercially available rabbit-ATG (Thymoglobulin, Sanofi).

Commercial anti-thymocyte globulin (ATG, (Thymoglobulin, Sanofi)) is useful for inducing transplant tolerance and is manufactured by immunizing New Zealand rabbits with human thymocytes; the blood is harvested from thousands of animals and antibodies are purified from the plasma. The library of ABPs, .i.e., rhATG, disclosed herein combines the efficacy advantages of a polyclonal ATG with the safety advantages of a fully human, recombinant ABP library.

First, transgenic mice carrying inserted human immunoglobulin genes were immunized with human thymocytes or human T cells. Footpad injections were performed on two Trianni Mice twice weekly for three weeks, followed by boosts the following two weeks. One to two million thymocytes were injected into each mouse at each timepoint. Before the final boosts, the serum titer of thymocyte antibodies was assessed by flow cytometry, using a dilution series of each animal's serum, starting at 1:200 and ending at 1:145,000. We observed a strong serum response in both animals, with one animal showing a slightly stronger response. Lymph nodes (popliteal, inguinal, axillary, and mesenteric) were surgically removed after sacrifice. Single cell suspensions for each animal were made by manual disruption followed by passage through a 70 μm filter. Next, we used the EasySep™ Mouse Pan-B Cell Isolation Kit (Stemcell Technologies) negative selection kit to isolate B cells from each sample. The lymph node B cell populations were quantified by counting on a C-Chip hemocytometer (Incyto) and assessed for viability using Trypan blue. The cells were then diluted to 5,000-6,000 cells/mL in phosphate-buffered saline (PBS) with 12% OptiPrep™ Density Gradient Medium (Sigma). This cell mixture was used for microfluidic encapsulation. We ran approximately one million B cells from each of the six animals through our emulsion droplet microfluidics platform.

A DNA library encoding scFv from RNA of single cells, with native heavy-light Ig pairing intact, was generated using the emulsion droplet microfluidics platform or vortex emulsions. The method for generating the DNA library was divided into 1) poly(A)+mRNA capture, 2) multiplexed overlap extension reverse transcriptase polymerase chain reaction (OE-RT-PCR), and 3) nested PCR to remove artifacts and add adapters for deep sequencing or yeast display libraries. The scFv libraries are generated from approximately one million B cells from each animal that achieved a positive titer.

For poly(A)+mRNA capture, a custom designed co-flow emulsion droplet microfluidic chip fabricated from glass (Dolomite) was used. The microfluidic chip has two input channels for fluorocarbon oil (Dolomite), one input channel for the cell suspension mix described above, and one input channel for oligo-dT beads (NEB) at 1.25 mg/ml in cell lysis buffer (20 mM Tris pH 7.5, 0.5 M NaCl, 1 mM ethylenediaminetetraacetic acid (EDTA), 0.5% Tween-20, and 20 mM dithiothreitol). The input channels are etched to 50 μm by 150 μm for most of the chip's length, narrow to 55 μm at the droplet junction, and were coated with hydrophobic Pico-Glide (Dolomite). Three Mitos P-Pump pressure pumps (Dolomite) were used to pump the liquids through the chip. Droplet size depends on pressure, but typically droplets of ~45 μm diameter were optimally stable. Emulsions were collected into chilled 2 ml microcentrifuge tubes and incubated at 40° C. for 15 minutes for mRNA capture. The beads were extracted from the droplets using Pico-Break (Dolomite). In some embodiments, similar single cell partitioning emulsions are made using a vortex.

For multiplex OE-RT-PCR, glass Telos droplet emulsion microfluidic chips were used (Dolomite). mRNA-bound beads were re-suspended into OE-RT-PCR mix and injected into the microfluidic chips with a mineral oil-based surfactant mix (available commercially from GigaGen) at pressures that generate 27 μm droplets. The OE-RT-PCR mix contains 2× one-step RT-PCR buffer, 2.0 mM MgSO₄, SuperScript III reverse transcriptase, and Platinum Taq (Thermo Fisher Scientific), plus a mixture of primers directed against the IgK C region, the IgG C region, and all V regions. The overlap region is a DNA sequence that encodes a Gly-Ser rich scFv linker sequence. The DNA fragments were recovered from the droplets using a droplet breaking solution (available commercially from GigaGen) and then purified using QIAquick PCR Purification Kit (Qiagen). In some embodiments, similar OE-RT-PCR emulsions were made using a vortex.

For nested PCR, the purified OE-RT-PCR product was first run on a 1.7% agarose gel for 80 minutes at 150 V. A band at 1200-1500 base pair (bp) corresponding to the linked product was excised and purified using NucleoSpin Gel and PCR Clean-up Kit (Macherey Nagel). PCR was then performed to add adapters for Illumina sequencing or yeast display; for sequencing, a randomer of seven nucleotides is added to increase base calling accuracy in subsequent next generation sequencing steps. Nested PCR was performed with 2×NEBNext High-Fidelity amplification mix (NEB) with either Illumina adapter containing primers or primers for cloning into the yeast expression vector. The nested PCR product was run on a 1.2% agarose gel for 50 minutes at 150 V. A band at 800-1100 bp was excised and purified using NucleoSpin Gel and PCR Clean-up Kit (Macherey Nagel).

To convert the GigaLink™ scFv libraries into full-length CHO expression libraries, nested outer PCR primers were used to add adapters with overhangs for Gibson assembly to the 5′ and 3′ ends of the scFv library. Then NEBuilder HiFi DNA Assembly Master Mix (NEB, Ipswich, MA, USA) was used to insert the scFv library into a vector containing a single promoter, a secretory leader sequence for light chain Ig and the remainder of the IgG1 constant region, creating a cloned scFv library. This intermediate library was transformed into E. coli, spread onto LB-ampicillin plates, 0.5-1 million colonies were scraped and pooled for a plasmid purification using ZymoPURE II Plasmid Maxiprep Kits (Zymo Research, Irvine, CA, USA). To create the full-length antibody library, a second Gibson assembly was performed by linearizing the product of GA1 with BamHI-HF (NEB, Ipswich, MA, USA) and using it as a vector to insert a synthetic amplicon containing a portion of the light chain Ig constant region, a poly(A) signal for light chain Ig, a promoter for the IgG gene and a secretory leader sequence for the IgG gene. The full-length library was then transformed into E. coli and spread on LB-ampicillin plates Over 0.5 million colonies are scraped and plasmid is purified with a ZymoPURE II Plasmid Maxiprep Kits (Zymo Research) to make the full-length recombinant hyperimmune maxiprep library for transfection.

The adherent Flp-In™-CHO cell line was adapted with a genomically integrated FRT site (Thermo Fisher Scientific, Waltham, MA, USA) to suspension culture. For all steps in the adaptation process, “Ham's F-12” refers to Ham's F-12 (with L-glutamine, Thermo Fisher Scientific, Waltham, MA, USA) plus 10% FBS (Thermo Fisher Scientific, Waltham, MA, USA) and “BalanCD” refers to BalanCD CHO Growth A (Irvine Scientific) with 4 mM Glutamax (Thermo Fisher Scientific, Waltham, MA, USA). To adapt this cell line to suspension, the cells were first passaged into a mixture of 50% Ham's F-12 plus 50% BalanCD in T-flasks. Cells were next passaged into 25% Ham's F-12 plus 75% BalanCD and switched to shaking Erlenmeyer flasks. Cells were then passaged into 10% Ham's F-12, 90% BalanCD+0.2% anti-clumping agent (Irvine Scientific, Santa Ana, CA, USA) and banked for future use.

100 million of the adapted Flp-In CHO cells were transfected per recombinant hyperimmune library using an Amaxa Nucleofector 4D (SG buffer, pulse DU133; Lonza, Basel, Switzerland). These cells were plated into shaking Erlenmeyer flasks and recovered in an incubator at 37° C. and 125 rpm for 48 hours. After 48 hours, the cells were counted to determine viability, cells were seeded at 1 million cells/mL, and selection was started using 600 μg/mL Hygromycin-B (Gemini Bio, West Sacramento, CA, USA) in fresh media. Cells were counted and media was changed every 2-3 days during the 7-day selection. The libraries were kept on 600 μg/mL Hygromycin-B (Gemini Bio, West Sacramento, CA, USA) during expansion until viability exceeded 95%. When cells were >95% viable and doubling every 24 hours, the cell line was banked for liquid nitrogen storage.

CHO cells stably expressing antibody libraries were grown in media consisting of 90% BalanCD CHO Growth A Medium (Irvine Scientific, Santa Ana, CA), 9% Ham's F-12 (Thermo Fisher Scientific, Waltham, MA, USA), 1% FBS (ThermoFisher Scientific), 4 mM Glutamax (Thermo Fisher Scientific, Waltham, MA, USA), 0.2% anti-clumping agent (Irvine Scientific, Santa Ana, CA, USA). For small-scale production, cells were seeded at 1×10⁶ cells/mL into 50 mL media in a 250 mL Erlenmeyer flask and grown at 37° C., 5% CO₂, 125 rpm. Cells were continually grown under these conditions and supplemented with 7.5 mL CHO Feed 1 (Irvine Scientific, Santa Ana, CA, USA) on days 2, 4 and 7 of the production run. Supernatant was harvested on Day 8 by centrifugation followed by filtration through a 0.22 μm 250 mL filter bottle (EMD Millipore, Burlington, MA, USA) with 1 μm pre-filter (EMD Millipore, Burlington, MA, USA). Harvested cell culture fluid (HCCF) was stored at 4° C. until Protein A purification. For large-scale production of the plasma cell recombinant hyperimmune, cells were grown in the same media but with some modifications to the production conditions. A seed train was used to scale the cultures from 2×10⁷ cells to 1.2×10¹⁰ cells at 37° C. Cells were then seeded at 1×10⁶ cells/mL in 2 L in a 5 L flask (in triplicate; Day 0). On Day 2 the temperature was shifted from 37° C. to 33° C. Each flask was fed with 300 mL CHO Feed 1 (Irvine Scientific, Santa Ana, CA, USA) on days 2, 4, 6, 8, 10, and 13 of the culture. Supernatant was harvested on Day 14.

After harvest, HCCF was purified with MabSelect SuRe Protein A resin (GE Life Sciences, Marlborough, MA, USA) using the following buffers: Equilibration, Chase, Wash 2 (25 mM Tris, 150 mM NaCl, pH 7.4), Wash 1 (25 mM Tris, 1 M NaCl, pH 7.4), Elution (20 mM citric acid, pH 3.0), Neutralization (100 mM Tris, pH 8.0 for small scale, 1 M Tris, pH 9.0 for large scale). The column was sanitized before and after use with 0.1 N NaOH. For the large-scale production of the plasma cell recombinant hyperimmune, an additional Wash 3 consisting of 0.5 M arginine, pH 7.4 was used, followed by an additional wash with Wash 2 before elution. The order of purification steps was: Equilibration, Load, Chase, Wash 1, Wash 2, (large scale: Wash 3, Wash 2), Elution, Neutralization (added manually into tubes used for collection of eluate fractions). The recombinant hyperimmunes (ABPs) were concentrated using Vivaspin 20, 30 kDa molecular weight cut off spin concentrators (Sartorius, Göttingen, Germany) and formulated in PBS (small-scale productions) or 0.2 M glycine, pH 4.5 (large scale production), followed by 0.22 μm filtration.

ELISA was used to test binding of the rhATG, i.e., anti-T cell and anti-thymocyte ABPs against antigens known to be expressed on the surface of T cells and thymocytes. ELISA showed binding to CD4, CD45, and CD81. Antigens were coated on an ELISA plate at 1 ug/mL. Titration curves were performed starting at 100 ug/mL of each antibody with a 1/3 stepwise dilution to determine the EC50. Because different secondary detection antibodies were used, the EC50 values cannot be directly compared between rabbit-ATG and rhATG. However, it was determined that within each library the antigens had stronger binding than their respective background. Antibody responses were broadly reactive against many T cell antigens for both rhATG and rabbit-ATG, with both binding very strongly to CD45 and CD5, and binding weaker to CD4, CD11, and CD81 (data not shown).

An in vivo validation study was performed. An in vivo model of GvHD (graft-versus-host-disease) was used to demonstrate the functional efficacy of ATG treatment-induced delay to GvHD. 1×10\hat{ }7 human PBMCs from a single donor were engrafted into NSG mice. The study used 6 mice per group with an IV infusion of the drugs tested: rhATG (ABPs), commercial rabbit-ATG, and a vehicle control. Animals were treated (6 mg/kg) at a single timepoint 7 days after engraftment. Additionally, a positive control group (8 mice) received Abatacept, a drug commonly used to prevent GvHD, and this was dosed intraperitoneally (IP) every other day from day 5 to the end of study. Immune cells were measured by flow cytometry for expansion, denoting progression to GvHD, and animals were monitored for weight loss and clinical presentation of GvHD leading to death.

Forty-two days after PBMC engraftment, any animals that were still alive were taken down and a survival analysis was completed for each of the treatment groups. There was no significant delay with rhATG (p=0.2, Mantel-Cox) and only a minor delay to GvHD was observed with rabbit-ATG (p=0.01, Mantel-Cox) (data not shown). Flow cytometry was used to measure engrafted PBMCs before treatment, 2 days after treatment, and 9 days after treatment. rhATG and rabbit-ATG depleted CD45+ cells, as seen 2 days after treatment, leading to a delay in the full engraftment of CD45+ cells, however by day 9 there with no significant difference between any groups (data not shown).

The results demonstrate that the rhATG (library of ABPs) has a similar antigen-specific antibody binding profile as the currently available commercial rabbit-ATG, though some differences were observed. In addition, rhATG also performs similarly to commercial rabbit-ATG in delaying progression to GvHD in mice using different dosing regimens.

### 7.1.2. Example 2: Generation of a Library of ABPs with Activity Against Haemophilus influenzae Type b (Hib) from Human Donors

Both in vitro and in vivo studies pere performed, testing polyclonal antibody pools, (pAb), i.e., libraries of ABPs, with activity against Haemophilus influenzae type b (Hib). Tested were anti-Hib pAbs made from four different B cell subtypes collected from donors vaccinated with the Pedvax-HIB conjugate vaccine. The four subtypes tested were CD43⁺ plasmablasts, CD27+ memory B cells, peripheral CD138⁺ plasma cells, and pan-B cells (all B cells). All four pAbs were first tested in vitro. The pAb made from CD138⁺ plasma cells was the most potent in vitro, so this product was then tested relative to IVIG in an in vivo challenge model.

A CRO (BloodCenter Wisconsin, Milwaukee, WI, USA) was used to vaccinate two donors (Donor 1, a 26-year-old Caucasian female, and Donor 2, a 21-year-old Asian male) with PedvaxHIB vaccine (Merck, Kenilworth, NJ, USA). Leukapheresis was performed eight or nine days later to obtain PBMCs. In parallel, plasma was isolated from separate blood draws on the day of leukapheresis and prior to vaccination. ELISA against Hib (Alpha Diagnostics, San Antonio, TX, USA; see methods below) on the plasma samples confirmed a response to the vaccine as compared to plasma from the same donors prior to vaccination. Sample collection protocols were approved by Institutional Review Board (IRB) protocol #PRO00028063 (Medical College of Wisconsin/Froedtert Hospital IRB) to GigaGen. Informed consent was obtained from all participants and samples were shipped to GigaGen de-identified.

To isolate pan-B cells, we used the Human EasySep Pan-B Cell Enrichment Kit (Stemcell #19554, Vancouver, BC, Canada). To isolate CD43⁺ cells, we used the pan-B cells and positive selection beads for CD43 (Miltenyi #130-091-333, Bergisch Gladbach, Germany). To isolate CD27⁺ cells, we applied CD27 positive selection beads (Miltenyi #130-051-601, Bergisch Gladbach, Germany) to the negative fraction from the CD43⁺ selection. For plasma cells, we applied the EasySep Human CD138 Positive Selection Kit (Stemcell #18357, Vancouver, BC, Canada) to PBMCs. After isolation, the antibody-producing cells were cryopreserved using CryoStor® CS10 (Stemcell Technologies, Vancouver, BC, Canada). Immediately prior to generating paired heavy and light chain libraries, cells were thawed, washed in cold DPBS+0.5% BSA, assessed for viability with Trypan blue on a Countess™ cell counter (Thermo Fisher Scientific, Waltham, MA, USA), and then re-suspended in 12% OptiPrep™ Density Gradient Medium (Sigma, St. Louis, MO, USA) at 5,000-6,000 cells per μl. This cell mixture was used for microfluidic encapsulation as described in the next section.

Generation of scFv libraries from antibody-producing cells (Adler et al., Mabs 9, 1282-1996, 2017) comprises three steps: (i) poly(A)+mRNA capture, (ii) multiplexed overlap extension reverse transcriptase polymerase chain reaction (OE-RT-PCR), and (iii) nested PCR to remove artifacts and add adapter sequences for deep sequencing or yeast display libraries.

To convert the GigaLink™ scFv libraries into full-length CHO expression libraries, we first used nested outer PCR primers to add adapters with overhangs for Gibson assembly to the 5′ and 3′ ends of the scFv library. Then NEBuilder HiFi DNA Assembly Master Mix (NEB, Ipswich, MA, USA) was used to insert the scFv library into a vector containing a single promoter, a secretory leader sequence for light chain Ig and the remainder of the IgG1 constant region, creating a cloned scFv library. This intermediate library was transformed into E. coli, spread onto LB-ampicillin plates, 0.5-1 million colonies were scraped and pooled for a plasmid purification using ZymoPURE II Plasmid Maxiprep Kits (Zymo Research, Irvine, CA, USA). To create the full-length antibody library, we performed a second Gibson assembly by linearizing the product of GA1 with BamHI-HF (NEB, Ipswich, MA, USA) and using it as a vector to insert a synthetic amplicon containing a portion of the light chain Ig constant region, a poly(A) signal for light chain Ig, a promoter for the IgG gene and a secretory leader sequence for the IgG gene. The full-length library was then transformed into E. coli and spread on LB-ampicillin plates. We typically scrape >0.5 million colonies and purify plasmid with a ZymoPURE II Plasmid Maxiprep Kits (Zymo Research) to make the full-length recombinant hyperimmune maxiprep library for transfection.

We adapted the adherent Flp-In™-CHO cell line with a genomically integrated FRT site (Thermo Fisher Scientific, Waltham, MA, USA) to suspension culture. For all steps in the adaptation process, “Ham's F-12” refers to Ham's F-12 (with L-glutamine, Thermo Fisher Scientific, Waltham, MA, USA) plus 10% FBS (Thermo Fisher Scientific, Waltham, MA, USA) and “BalanCD” refers to BalanCD CHO Growth A (Irvine Scientific) with 4 mM Glutamax (Thermo Fisher Scientific, Waltham, MA, USA). To adapt this cell line to suspension, we first passaged the cells into a mixture of 50% Ham's F-12 plus 50% BalanCD in T-flasks. Cells were next passaged into 25% Ham's F-12 plus 75% BalanCD and switched to shaking Erlenmeyer flasks. Cells were then passaged into 10% Ham's F-12, 90% BalanCD+0.2% anti-clumping agent (Irvine Scientific, Santa Ana, CA, USA) and banked for future use.

100 million of the adapted Flp-In CHO cells were transfected per recombinant hyperimmune library using an Amaxa Nucleofector 4D (SG buffer, pulse DU133; Lonza, Basel, Switzerland). These cells were plated into shaking Erlenmeyer flasks and recovered in an incubator at 37° C. and 125 rpm for 48 hours. After 48 hours, the cells were counted to determine viability, cells were seeded at 1 million cells/mL, and selection was started using 600 μg/mL Hygromycin-B (Gemini Bio, West Sacramento, CA, USA) in fresh media. Cells were counted and media was changed every 2-3 days during the 7-day selection. The libraries were kept on 600 μg/mL Hygromycin-B (Gemini Bio, West Sacramento, CA, USA) during expansion until viability exceeded 95%. When cells were >95% viable and doubling every 24 hours, the cell line was banked for liquid nitrogen storage.

CHO cells stably expressing antibody libraries were grown in media consisting of 90% BalanCD CHO Growth A Medium (Irvine Scientific, Santa Ana, CA), 9% Ham's F-12 (Thermo Fisher Scientific, Waltham, MA, USA), 1% FBS (ThermoFisher Scientific), 4 mM Glutamax (Thermo Fisher Scientific, Waltham, MA, USA), 0.2% anti-clumping agent (Irvine Scientific, Santa Ana, CA, USA). For small-scale production, cells were seeded at 1×10⁶ cells/mL into 50 mL media in a 250 mL Erlenmeyer flask and grown at 37° C., 5% CO₂, 125 rpm. Cells were continually grown under these conditions and supplemented with 7.5 mL CHO Feed 1 (Irvine Scientific, Santa Ana, CA, USA) on days 2, 4 and 7 of the production run. Supernatant was harvested on Day 8 by centrifugation followed by filtration through a 0.22 μm 250 mL filter bottle (EMD Millipore, Burlington, MA, USA) with 1 μm pre-filter (EMD Millipore, Burlington, MA, USA). Harvested cell culture fluid (HCCF) was stored at 4° C. until Protein A purification. For large-scale production of the plasma cell recombinant hyperimmune, cells were grown in the same media but with some modifications to the production conditions. A seed train was used to scale the cultures from 2×10⁷ cells to 1.2×10¹⁰ cells at 37° C. Cells were then seeded at 1×10⁶ cells/mL in 2 L in a 5 L flask (in triplicate; Day 0). On Day 2 the temperature was shifted from 37° C. to 33° C. Each flask was fed with 300 mL CHO Feed 1 (Irvine Scientific, Santa Ana, CA, USA) on days 2, 4, 6, 8, 10, and 13 of the culture. Supernatant was harvested on Day 14.

After harvest, HCCF was purified with MabSelect SuRe Protein A resin (GE Life Sciences, Marlborough, MA, USA) using the following buffers: Equilibration, Chase, Wash 2 (25 mM Tris, 150 mM NaCl, pH 7.4), Wash 1 (25 mM Tris, 1 M NaCl, pH 7.4), Elution (20 mM citric acid, pH 3.0), Neutralization (100 mM Tris, pH 8.0 for small scale, 1 M Tris, pH 9.0 for large scale). The column was sanitized before and after use with 0.1 N NaOH. For the large-scale production of the plasma cell recombinant hyperimmune, we used an additional Wash 3 consisting of 0.5 M arginine, pH 7.4, followed by an additional wash with Wash 2 before elution. The order of purification steps was: Equilibration, Load, Chase, Wash 1, Wash 2, (large scale: Wash 3, Wash 2), Elution, Neutralization (added manually into tubes used for collection of eluate fractions). The recombinant hyperimmunes were concentrated using Vivaspin 20, 30 kDa molecular weight cut off spin concentrators (Sartorius, Göttingen, Germany) and formulated in PBS (small-scale productions) or 0.2 M glycine, pH 4.5 (large scale production), followed by 0.22 μm filtration.

Imaged capillary isoelectric focusing (iCIEF) was performed using a Maurice imaging cIEF analyzer (Protein Simple, San Jose, CA, USA). Capillary electrophoresis sodium dodecyl sulfate (CE-SDS) was performed under reducing and non-reducing conditions using LabChip GX II Touch HT (Perkin Elmer, Waltham, MA, USA). Endotoxin levels were measured using Endosafe nexgen-PTS (Charles River, Wilmington, MA, USA).

We observed an HBV ABP yield of 92.2% in our Protein A step. Under non-reducing conditions, we observed a single peak (>99%) at 166.2 kDa with CE-SDS. Under reducing conditions, the ABP showed >99% pure IgG monomer and <1% other proteins, whereas plasma IVIg showed approximately 3.1% unknown protein, suggesting that recombinant hyperimmunes could be produced at higher purity of IgG than plasma IVIg. Analysis of the purified recombinant hyperimmune by iCIEF revealed a broad spectrum of isoelectric species, though plasma IVIg showed a considerably broader range of isoelectric species. We speculate that plasma IVIg has a broader variety of isolectric species because it comprises a broader diversity of antibodies, and also includes different IgG isotypes (the recombinant hyperimmune is only IgG1), as well as IgL. Finally, the endotoxin level was <0.5 endotoxin units (EU)/mg, which is the typical benchmark for recombinant mAb therapeutics.

Deep antibody sequencing libraries were quantified using a quantitative PCR Illumina Library Quantification Kit (KAPA, Wilmington, MA, USA) and diluted to 17.5 μM. Libraries were sequenced on a MiSeq (Illumina, San Diego, CA, USA) using a 500 cycle MiSeq Reagent Kit v2, according to the manufacturer's instructions. To make sequencing libraries, we used tailed-end PCR to add Illumina sequencing adapters to the 5′ and 3′ ends of the constructs of interest. Then, we obtained forward reads of 340 cycles and reverse reads of 162 cycles. This produced forward and reverse reads that overlap at the CDR3-H and part of the VH-gene, which increased confidence in nucleotide calls. Sequence analysis was performed using our previously reported bioinformatics pipeline (Adler et al., Mabs 9, 1282-1996, 2017). Pearson correlation was performed using the cor function in R version 3.4.2.

Each of four HBV ABPs were derived from 1.12-1.39 million input cells. After the repertoires were subjected to our library generation pipeline, the clonal diversity of the recombinant hyperimmunes were all less than 2,000 antibody clones (range: 880 to 1,659), capturing a considerable fraction of the input antibody diversity. All four recombinant hyperimmunes had a median germline IgHV identity of 93%, suggesting that no cell type yielded antibodies with significantly higher affinity, consistent with prior analysis of Hib-vaccinated individuals (Truck et al., 2015). Clonal diversity was not strongly biased toward the most frequent antibodies in any of the mixtures. The most common antibody was present at a frequency of 3.5% (plasma cell hyperimmune). The pan-B recombinant hyperimmune had the least skewed clonal diversity (the top 20 antibodies were 12.7% of all antibodies), and the plasma cell recombinant hyperimmune had the most skewed clonal diversity (the top 20 antibodies were 26.6% of all antibodies).

We examined the genetic diversity of the four recombinant hyperimmune libraries. An overlap analysis revealed that no more than 11.8% of clones were shared between any given two recombinant hyperimmune libraries. Pearson correlation analysis was not significant between any two pairwise comparisons (p<0.01). All four recombinant hyperimmune libraries contained a variety of IgGV-J gene pairings, including high frequencies of antibodies with IgHV3-23 and IgHJ4 genes, which has been seen elsewhere in anti-Hib repertoires (Silverman & Lucas, 1991; Adderson et al., 1993; Lucas et al., 2003; Trück et al., 2015). Other common IgHV genes included IgHV3-30, IgHV1-69, and IgHV3-7. All libraries also included complementarity-determining region (CDR) 3 sequences containing either of the peptides GYGFD or GYGMD, previously observed in anti-Hib repertoires (Lucas et al., 2003; Trück et al., 2015). We conclude that all four libraries contain canonical anti-Hib sequences and similar levels of divergence from germline and genetic diversity. However, the four libraries do comprise distinct antibody mixtures, which may have different functional characteristics.

The Human Anti-Hib-PRP IgG ELISA kit (Alpha Diagnostics #980-100-PHG, San Antonio, TX, USA) was used for anti-Hib ELISA titers. Serial dilutions of antibody preparations were performed in Low NSB (non-specific binding) sample diluent. Quantitative measurements were performed on a plate reader (Molecular Devices, Fremont, CA, USA) at 450 nm. EC50 values were calculated using SoftMax Pro (Molecular Devices, Fremont, CA, USA). We also determined the anti-Hib PRP antibody titer for a pool of plasma from both donors before and after vaccination with the Hib active vaccine, as well as IVIg. The plasma cell, pan-B, and plasmablast recombinant hyperimmunes yielded considerably higher Hib-binding titers than IVIg (range: 160× to 2,323×), with the plasma cell hyperimmune yielding the highest titer. The post-vaccination plasma was only 3.7× the anti-Hib titer of IVIg, and no anti-Hib titer was detected in the memory B cell recombinant hyperimmune under the conditions tested. Taken together, these data indicate that our manufacturing process can considerably increase anti-Hib titers simply by selecting appropriate cell types from vaccinated donors.

In vitro neutralization studies were performed at a CRO (ImQuest Frederick, MD, USA). The Haemophilus influenzae type b Eagan strain was obtained from Zeptometrix (#0801679, Buffalo, NY, USA) as a frozen glycerol stock and stored at −80° C. The Haemophilus influenzae strain ATCC 10211 was obtained from the American Type Culture Collection (ATCC, Frederick, MD, USA) as a lyophilized stock and was propagated as recommended by the supplier. Colonies from an overnight incubation on chocolate agar plates were inoculated into growth media (Brain Heart Infusion, or BHI broth, BD BBL 299070, San Jose, CA, USA, with 2% Fildes enrichment, Remel #R45037, San Diego, CA, USA) and allowed to achieve an optical density of 625 nm (OD₆₂₅) of approximately 0.4. The culture was adjusted to an OD₆₂₅ of 0.15, which is equivalent to approximately 5×10⁸ colony forming units (CFU)/mL. The culture was further diluted to 5×10⁴ CFU/mL in dilution buffer (Hanks Balanced Salt Solution, Gibco, Waltham, MA, USA #14025-092, with 2% Fildes enrichment). The density of the bacterial culture used in the method was confirmed by plating 50 μL of the 5×103 and 5×102 dilutions in duplicate on chocolate agar and enumerating the colonies following incubation at 37° C./5% CO₂ for 24 hours.

Test articles were diluted three-fold in dilution buffer, starting at 200 μg/mL such that ten total dilutions were evaluated. 10 μL of each dilution of test article were added in duplicate to a 96-well microtiter plate. Eagan or ATCC 10211 bacteria at a concentration of approximately 5×10⁴ CFU/mL were then added to the plate in a volume of 20 μL, such that the total in-well bacterial density would be 1×10⁴ CFU/20 μL. Following an incubation of 15 minutes at 37° C./5% CO₂, 25 μL of baby rabbit complement (Pel-Freez #31061-1, Rogers, AR, USA) and 25 μL of dilution buffer was added to each well. The plate was incubated at 37° C./5% CO₂ for 60 minutes. Following the incubation, 5 μL of each reaction mixture was diluted in 45 μL of dilution buffer and the entire 50 μL was plated on chocolate agar plates. The plates were incubated for approximately 16 hours at 37° C./5% CO₂. Following incubation, bacterial colonies were enumerated. The test article concentration that killed>50% of the bacteria is the SBI.

As expected from the ELISA data, the memory B cell recombinant hyperimmune was not able to neutralize either Hib strain at any of the concentrations tested. The plasma cell recombinant hyperimmune again yielded the highest titer, with SBIs of 81 and 243 for the Eagan and ATCC10211 strains, respectively. The pan-B and plasmablast recombinant hyperimmunes were 1/9^{th} as potent as the plasma cell recombinant hyperimmune. Neutralization was not detected for IVIg at any of the tested concentrations. We conclude that the plasma cell recombinant hyperimmune is the highest potency among the four cell types tested.

All vertebrate experiments were conducted under supervision and approval of either the Institutional Animal Care and Use Committee of Sinclair Research Center, LLC, Missouri (USA) in accordance with the Animal Welfare Act and standards incorporated in the Guide for the Care and Use of Laboratory Animals (National Research Council of the National Academies, Eighth Edition) or the National Committee of Animal Ethics, Denmark, in accordance with the standards of EU Directive 2010/63/EU (permission number: 2014-15-0201-00171).

For acute toxicity, Balb/cJ mice (Charles River, Wilmington, MA, USA) were divided randomly by a CRO (Sinclair Research, Auxvasse, MO, USA) into seven groups of six animals per group. Three of the groups were administered the recombinant hyperimmune at a single dose of 30 mg/kg, 100 mg/kg, or 300 mg/kg. A negative control group was administered a single dose of saline vehicle. The three remaining groups were administered a single dose of plasma IVIg (Gammagard; Grifols, Sant Cugat, Catalonia) at 30 mg/kg, 100 mg/kg, or 300 mg/kg. Test article samples were diluted in 0.2 M Glycine, pH 4.5. Test article administration was performed intravenously through a tail vein. Dose volumes were calculated based on each individual animal's most recent body weight. The mice were then observed twice daily for 8 days for general health, reaction at the site of test article administration, morbidity and mortality, body weight, and gross physical examination (skin, mucous membranes, eyes, ears, nose, and respiration). Animals were euthanized with CO₂ gas after 3 days, and terminal serum chemistry was performed, including albumin, globulin, glucose, total protein, blood urea nitrogen, and several other metrics.

We observed no test article-related findings for any of the test groups. We conclude that the no-observed-adverse-effect level (NOAEL) for a single intravenous dose of the plasma cell recombinant hyperimmune is 300 mg/kg. IVIg is typically dosed in immunodeficient patients at around 300 mg/kg for protection against Hib and other pathogens, and the Hib hyperimmune product is thousands-fold more potent, so we conclude that the plasma cell recombinant hyperimmune would have no observable toxicity for a minimally efficacious dose.

For pharmacokinetics, a CRO (Sinclair Research, Auxvasse, MO, USA) administered twenty male Balb/cJ mice (Charles River, Wilmington, MA, USA) one 100 mg/kg intravenous tail vein dose of the plasma cell recombinant hyperimmune. A sparse blood sampling procedure was followed such that no mice received more than two of the scheduled seven PK blood samplings. We then used a sandwich ligand-binding method (LBA) and Meso Scale Discovery (MSD; Rockville, MD, USA) electrochemiluminescence (ECL) technology to measure serum human IgG. Capture antibody (SouthernBiotech #2049-01, Birmingham, AL, USA) was coated onto 96-well plates (MSD, Rockville, MD, USA). Serum samples were diluted to the minimum required dilution (MRD) of 1:100 in PBS/T containing 1% BSA (PBS/T/BSA). Next, the diluted samples were added to the designated wells. After another wash step, wells were inoculated with PBS/T/BSA containing 1 mg/mL of biotinylated-goat anti-human IgG (SouthernBiotech #2049-08, Birmingham, AL, USA). After incubation, streptavidin-SULFO-TAG was added, followed by 2× read buffer T (MSD, Rockville, MD, USA). ECL units were measured using an MSD QuickPlex SQ 120 instrument. A standard curve was additionally generated for each run using plasma cell-based recombinant hyperimmune. The Discovery Workbench software (MSD, Rockville, MD, USA) was used to fit the data using a four-parameter logistic (4-PL) curve-fit of mean ECL units versus nominal IgG standard values. We removed from further analysis two animals with 1100 ng/ml or lower readings at the 1-hour timepoint, under the assumption that intravenous administration failed. We then used the PKNCA package in R (Denney et al., 2015) to apply non-compartmental analysis to the concentration-time data to estimate maximum observed plasma concentration (Cₘₐₓ), time of maximum observed plasma concentration (Tₘₐₓ), and half-life (t_{1/2}).

The maximum observed plasma concentration was 12,360 ng/ml (Cₘₐₓ), observed one hour post-dose (Tₘₐₓ). The half-life (t_{1/2}) of the recombinant hyperimmune was approximately 34.5 hours. Combining these data with the ELISA titer data, we estimate that the maximum anti-Hib trough level was 861 IU/mL for a single 100 mg/kg intravenous dose.

The Haemophilus influenza strain ATCC10211 was grown on chocolate agar plates overnight at 35° C. and 5% CO₂. Single overnight colonies were resuspended in sterile saline to 1.5×10⁸ CFU/mL. This suspension was diluted in BHI broth with 5% mucin and 2% hemoglobin to approximately 1×10⁶ CFU/mL and further 10-fold diluted to 10 CFU/mL.

Balb/cJ mice (Taconic, Denmark; n=6 per group) were inoculated with single 0.5 mL intraperitoneal doses of 104, 105, or 10⁶ CFU/mL Hib bacteria (strain ATCC10211). Approximately 1 hour before inoculation, mice were treated orally with 45 □L Nurofen (20 mg ibuprofen/mL corresponding to approximately 30 mg/kg) as pain relief. Twenty-four hours prior to inoculation, mice were administered 300 mg/kg recombinant Hib hyperimmune, 300 mg/kg plasma IVIg or saline. One hour after inoculation mice were dosed with 20 mg/kg ciproflaxin antibiotic as positive control treatment. Mice were scored for clinical signs of infection every 2-6^{th} hour and were terminated when severely affected by the infection. After another 72 hours, any living animals were anesthetized with Zoletil mix and blood was collected by axillary cut down. Mice were sacrificed by cervical dislocation, 2 mL sterile saline was injected intraperitoneally, and the abdomen gently massaged before it was opened and fluid sampled with a pipette. Each sample was 10-fold diluted in saline and 20 μL spots were applied on chocolate agar plates. All agar plates were incubated 18-22 hours at 35° C. at ambient air.

The Hib infection was lethal to all but one mouse at all inoculation doses in the vehicle control group. In contrast, only one out of 18 mice was severely affected in the recombinant hyperimmune treatment groups (in the 10⁶ CFU inoculation group). IVIg was much less protective than the recombinant hyperimmune, with 5/6 mice in the 10⁵ CFU and 10⁶ CFU inoculation groups, and 2/6 mice in the 10⁴ CFU inoculation group being severely affected by the infection. Analysis of bacterial loads in blood demonstrated that the recombinant hyperimmune eliminated Hib from the bloodstream of all animals, whereas IVIg treatment resulted in significantly lower bacterial loads than the vehicle control in only one of the inoculation groups and no significant reduction in two inoculation groups (Dunnett's multiple comparisons test, p<0.05). In peritoneal lavage, the recombinant hyperimmune again significantly reduced the bacterial loads compared to the vehicle control group (Dunnett's multiple comparisons test, p<0.05). However, whereas Hib bacteria were not detectable in the peritoneal lavage of surviving animals treated with ciproflaxin, Hib bacteria were detectable in the peritoneal lavage of 6/17 surviving animals treated with recombinant hyperimmune (range: 23-77 CFU/mL). This suggests differences in the efficacy of the recombinant hyperimmune between the peritoneum and blood, perhaps due to bioavailability of drug or complement in the peritoneum.

In some embodiments, the Hib hyperimmune is spiked into conventional plasma IVIg to increase the anti-Hib titer of IVIg. In some embodiments, several anti-pathogen hyperimmunes are spiked into conventional plasma IVIg, for example, hyperimmunes directed against Hib, pneumococcus, influenza A virus, and tetanus are spiked into plasma IVIg to treat patients with primary immune deficiency. The spike in hyperimmunes increases the titer of antibodies directed against pathogens to which primary immune deficiency patients are particularly susceptible. Any number of spike-ins can be mixed with plasma IVIg to generate increased titers against any number of pathogens.

Using a series of in vitro and in vivo experiments, the following was determined. For Hib, plasma cells following vaccination produce the most potent ABPs. The plasma cell Hib ABPs was >2,300× more potent (by ELISA) than plasma IVIG. The plasma cell Hib ABPs strongly protected against Hib infection in an in vivo challenge model. Use of plasmablasts and pan-B cells also led to a potent ABPs in vitro, albeit less potent than plasma cells. For this antigen, the ABPs made from memory B cells had undetectable levels of potency in the in vitro methods.

### 7.1.3. Example 3: Generation of a Library of ABPs with Activity Against Streptococcus pneumoniae Capsular Polysaccharides

Streptococcus pneumoniae causes pneumococcal pneumonia. A recombinant polyclonal antibody (pAb), i.e., library of ABPs, with activity towards Streptococcus pneumoniae was generated, “GG-Pnc.” GG-Pnc tested in vitro. The results demonstrate the in vitro functional efficacy and potency of GG-Pnc with activity against Streptococcus pneumoniae capsular polysaccharides. The library was analyzed by bulk pneumococcal polysaccharide ELISA, serotype-specific ELISA, and serotype-specific opsonophagocytic methods.

Using the recombinant techniques described in examples 1 and 2, GG-Pnc, i.e., a library of ABPs was prepared. This library was prepared from three donors vaccinated with the Pneumovax-23 vaccine. Pneumovax-23 consists of capsular polysaccharides from 23 pneumococcal serotypes. All three donors showed an increase in titer against pneumococcal capsular polysaccharides after vaccination, as measured by ELISA. The rpAb was made from a mixture of all B cell subtypes isolated from the donors.

The Alpha Diagnostics ELISA measures bulk polysaccharide-specific antibody responses to the 23 pneumococcal polysaccharides found in the Pneumovax-23 vaccine and was used to measure an EC50 of the ABP library. An 8-step, 3-fold dilution series was performed, and a 4-point logistic analysis was performed to calculate the EC50. The ABP library GG-Pnc was ~100 times more potent than IVIG.

A serotype multiplex ELISA was performed to assess antibody diversity of the GG-Pnc ABP library compared to IVIG. Twenty pneumococcal serotypes were measured by ELISA. Using an international standard for pneumococcal-specific responses, antibody-specific responses in GG-Pnc and IVIG (Gamunex) were measured. GG-Pnc had a similar or higher concentration than IVIG against all serotypes except for serotype 6A.

Serotype-specific opsonophagocytosis methods were performed to assess antibody-induced killing function. Fourteen pneumococcal serotypes were measured by opsonophagocytosis responses using GG-Pnc and IVIG (Gamunex). Consistent with the multiplex-ELISA, GG-Pnc was similar to or more effective than IVIG for all serotypes except for 6A.

A serotype 2-specific ELISA was performed to determine the ability of GG-Pnc to bind to this serotype, since it was not included in the prior analysis, but it is an available option for an in vivo mouse model. An 8-step, 3-fold dilution series was performed, and a 4-point logistic analysis was used to calculate the EC50; only GG-Pnc had a value since IVIG had minimal binding to serotype 2, even at very high concentrations.

The GG-Pnc ABP library strongly bound a diverse set of pneumococcal serotypes and was able to neutralize all serotypes tested based on in vitro opsonophagocytosis methods. GG-Pnc was similar to or more potent than IVIG for all but one serotype (for both binding and killing), with no serotype-specific enrichment procedures performed and using all B cells isolated from the vaccinated donors. GG-Pnc also strongly bound to serotype 2.

### 7.1.4. Example 4: Generation of a Library of ABPs with Activity Against Influenza a Antigen

A library of ABPs with activity towards Influenza A antigen (ABP1) was generated using the recombinant methods described herein.

### 7.1.5. Example 5: Generation of a Library of ABPs with Activity Against Hepatitis B Virus Antigen

Two libraries of ABPs with activity against Hepatitis B virus antigen were generated using the recombinant methods described herein. CHO cells stably expressing antibody libraries with activity against Hepatitis B virus antigen were used for generation of CHO master cell bank (MCB).

One vial from the MCB was thawed for expansion of the seed train. EX-CELL Advanced CHO Fed-Batch Medium (MilliporeSigma, Burlington, MA) was used for cell expansion and passaging from cell bank vial thaw through the cell expansion process in shake flasks and a rocking motion (RM) bioreactor. EX-CELL Advanced CHO Fed-Batch Medium was used as the basal medium for both the 200 L SUB seed culture (N−1) and the 200 L fed-batch production bioreactor. EX-CELL Advanced CHO F1 Feed (MilliporeSigma) and CellVento 4Feed COMP (MilliporeSigma) were fed. Glucose levels were monitored daily starting on Day 2 and maintained above 4 g/L by supplementing with a 500 g/L Glucose Stock (prepared from powder, MilliporeSigma) to increase the concentration to 6 g/L as needed. Antifoam additions are used as needed to mitigate any foam buildup after Day 2. Bioreactors were harvested when the cell viability is ≤75% or on Day 16.

The bulk harvest was clarified and then processed downstream by a series of standard chromatography purification steps and viral reduction steps: (i) affinity chromatography, (ii) low pH virus inactivation, (iii) hydrophobic interaction chromatography or membrane filtration, (iv) multimodal anion exchange chromatography or membrane filtration, (v) multimodal cation exchange chromatography, (vi) anion exchange chromatography or membrane filtration, (vii) cation exchange chromatography, (viii) virus filtration, and/or (ix) ultrafiltration and/or diafiltration.

### 7.1.6. Example 6: Fingerprint-Like Method of a Library of ABPs

A library of ABPs with binding specifity to Hepatitis B virus antigen (rHBIG) contains 2306 unique V_{H} sequences and 147 unique V_{L} sequences. In total, the library contains over 2000 unique IgG clones. Accordingly, traditional methods adopted for analyzing individual antibodies (e.g., sequencing) cannot be used to test the compositions comprising thousands of antibodies. There is a need for a new method to characterize and analyze identity and consistency of the compositions throughout multiple lots and/or over time.

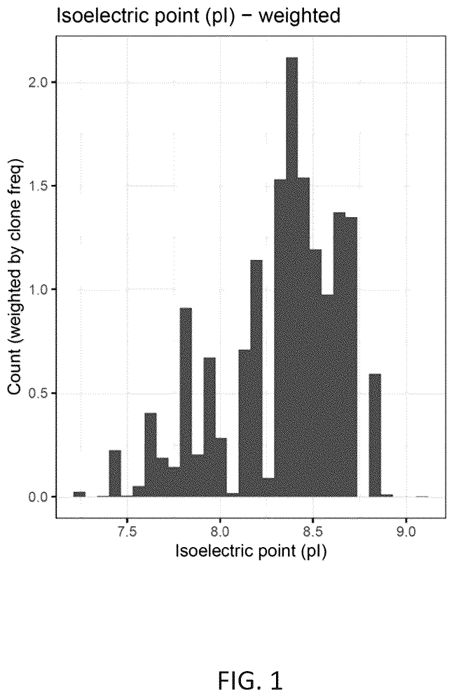

For development of a method, the sequences of the antibody clones in the library were analyzed and in silico bioinformatic analysis was performed to understand the distribution of charge states (isoelectric points) of the clones. The charge state distribution of the library, weighed by RNA-sequence abundance, is provided in FIG. 1.

The charge distribution was also determined experimentally. A novel CEX-HPLC (cation exchange high performance liquid chromatography) method was performed using a pH gradient for characterizing the charge distribution of the antibody population of recombinant pAb products. The CEX-HPLC profile of the rHBIG library showed multiple peaks, which is consistent with its polyclonal nature.

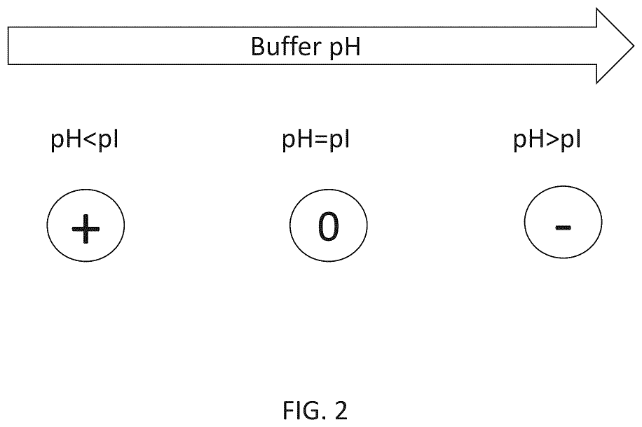

IgGs in the polyclonal library have different charge states, thus could be resolved by Cation Exchange-High Performance Liquid Chromatography (CEX-HPLC), involving a cation exchange chromatography with a pH gradient from a low pH (pH 5.6) mobile phase A to a high pH (pH 10.2) mobile phase B. As illustrated in FIG. 2, the polarity of the IgGs charges changes depending on the buffer pH.

The following materials and equipment were used for the CEX-HPLC.

Material and Equipment

| ITEM | VENDOR/PART NUMBER |
|---|---|
| Proteomix SCX NP1.7 4.6 × 100 mm | Sepax/401NP2-4610 |
| MAbPac SCX-10 HPLC Columns, | Thermofisher Scientific/082673 |
| 5μm, 4.6 × 250 mm | |
| CX-1 pH Gradient Buffer A (10X), | Thermofisher Scientific/302779 |
| 500 mL | |
| CX-1 pH Gradient Buffer B (10X), | Thermofisher Scientific /302780 |
| 500 mL | |
| Agilent HPLC 1260 or equivalent | NA |

HPLC Condition

| | |
|---|---|
| Mobile Phase A | CX-1 pH Gradient Buffer A, 1X (pH 5.6) |
| Mobile Phase B | CX-1 pH Gradient Buffer B, 1X (pH 10.2) |
| Column | Proteomix SCX NP1.7 4.6 × 100 mm |
| Flow Rate | 0.80 mL/min or specified |
| Column Temperature | 30° C. |
| Gradient Range | 10-100% B or specified |
| Gradient Time | 35 min or specified |

The method conditions were obtained by testing various conditions to maximize the resolution and minimize variation of the results (e.g., number of peaks over gradient length). Specifically, various flow rate, gradient of % B and gradient runtime were tested to achieve reproducibility, including column to column reproducibility and intra-method reproducibility.

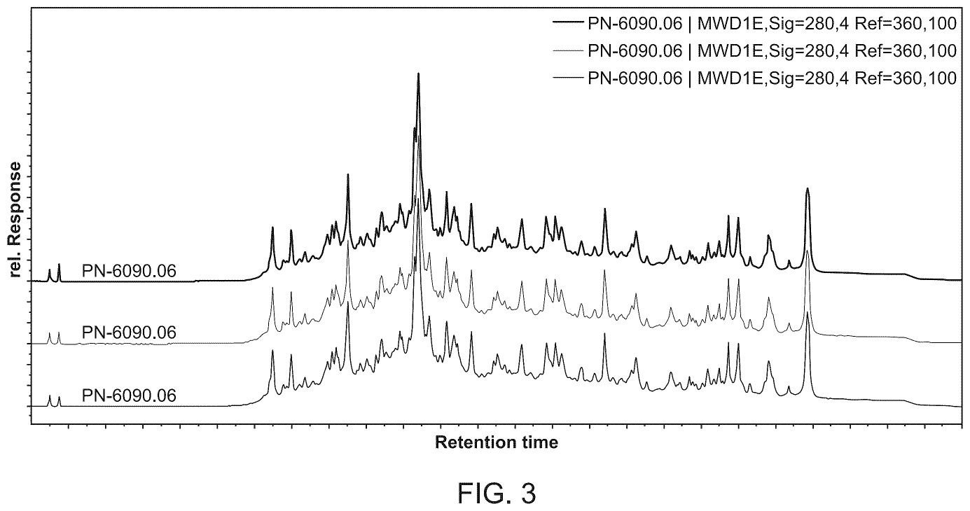

To test reproducibility of the method, the same batch of the polyclonal library was tested three times under the below condition and the results are provided in FIG. 3.

HPLC Condition

| | |
|---|---|
| Column | Proteomix SCX NP1.7 4.6 × 100 mm |
| Flow Rate | 0.80 mL/min |
| Column Temperature | 30° C. |
| Gradient Range | 10-100% B or specified |
| Gradient Time | 35 min |

FIG. 3 shows that the results (e.g., the peak area and height over the retention time (min)) are significantly consistent over the three separate runs showing high intra-method reproducibility.

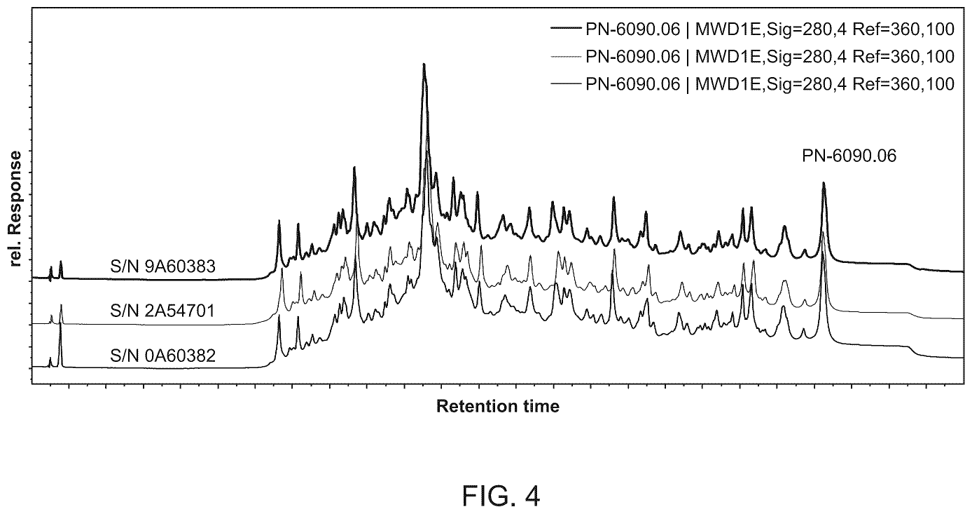

The CEX-HPLC method was performed using three different column lots-Proteomix SCX-NP1.7 (4.6×100 mm) columns: S/N 2A54701 (LN DW054), S/N 0A60382 (LN DW166), S/N 9A60383 (LN 430794). The three columns were installed at different ports and run on the same day. The results are provided in FIG. 4, which shows high column-to-column reproducibility.

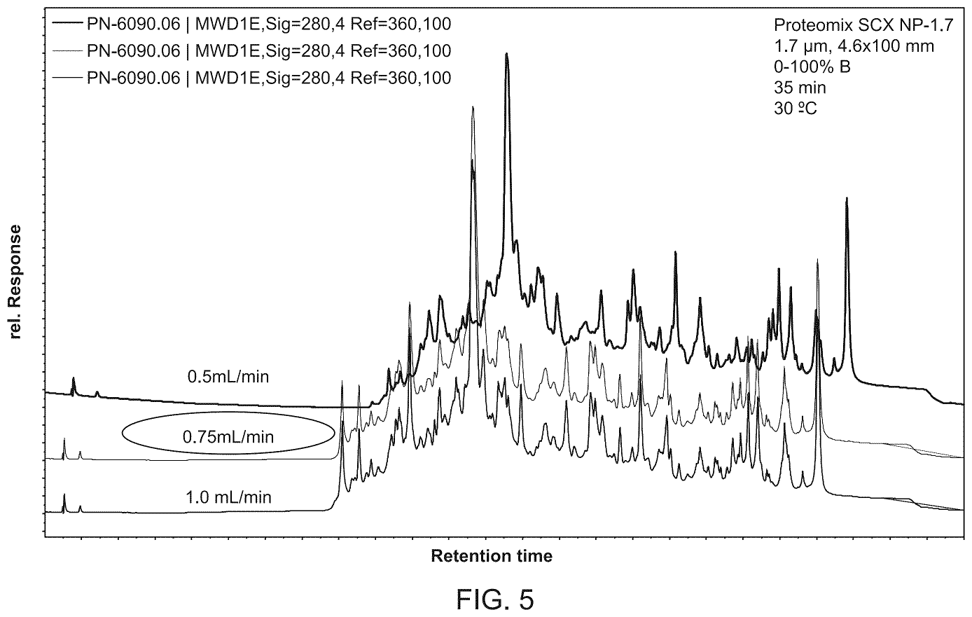

To find the optimal flow rate for resolution, the CEX-HPLC method was performed at three different flow rates (0.5 mL/min, 0.75 mL/min and 1.0 mL/min) under the below condition. The results provided in FIG. 5 show that flow rates of 0.75 mL/min and 1.0 mL/min provided comparable resolutions. A slightly lower flow rate (0.75 mL/min) was selected to maintain pressure in the optimal range.

HPLC Condition

| | |
|---|---|
| Column | Proteomix SCX NP1.7 4.6 × 100 mm |
| Column Temperature | 30° C. |
| Gradient Range | 0-100% B |
| Gradient Time | 35 min |

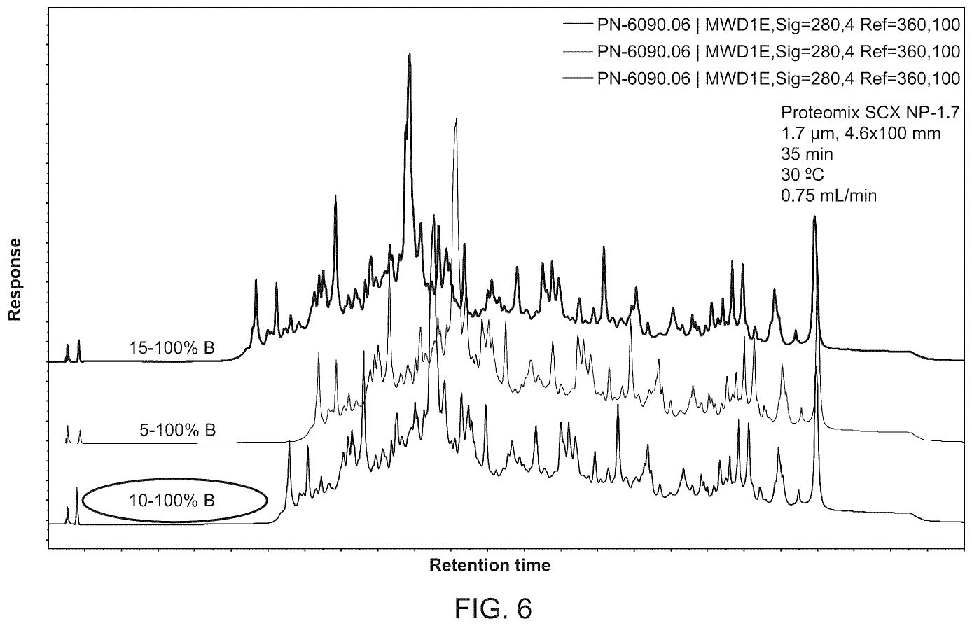

The CEX-HPLC method was performed at three different gradients (15-100% B, 5-100% B, and 10-100% B) under the below condition. The results provided in FIG. 6 show that narrower ranges tend to provide higher resolutions. The impact of sample pH was occasionally observed. The 10-100% B was selected to reduce sample matrix effect (pH).

HPLC Condition

| | |
|---|---|
| Column | Proteomix SCX NP1.7 4.6 × 100 mm |
| Column Temperature | 30° C. |
| Flow Rate | 0.75 mL/min |
| Gradient Time | 35 min |

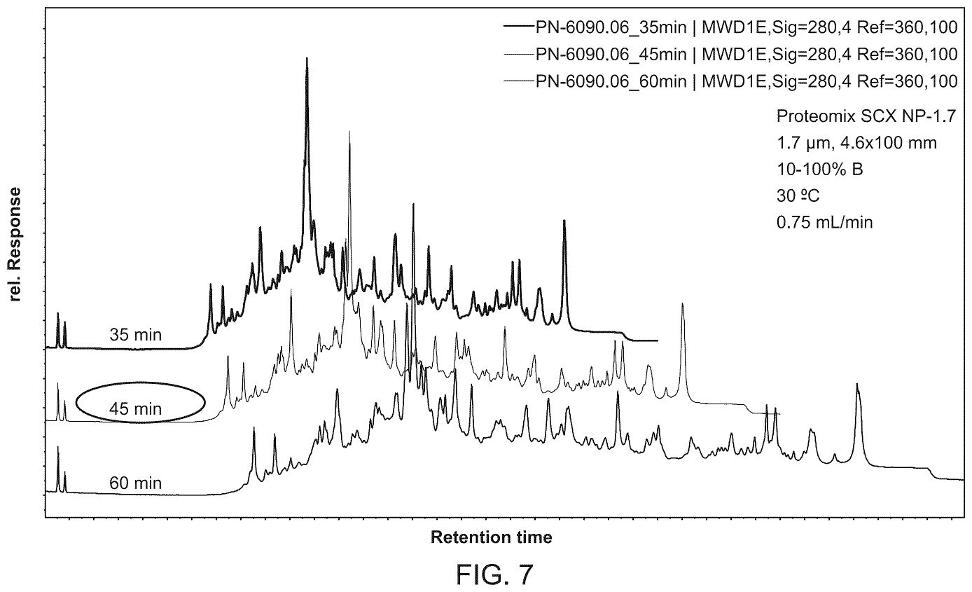

The CEX-HPLC method was performed at three different gradient times (35 min, 45 min and 60 min) under the below condition. The results provided in FIG. 7 show that longer gradient time tends to provide higher resolutions. 45 min gradient time was selected to achieve good resolution while maintaining reasonable runtime.

HPLC Condition

| | |
|---|---|
| Column | Proteomix SCX NP1.7 4.6 × 100 mm |
| Column Temperature | 30° C. |
| Flow Rate | 0.75 mL/min |
| Gradient Range | 0-100%B |

From the series of experiments, the following condition was selected for the CEX-HPLC method.

Optimized HPLC Condition

| | |
|---|---|
| Mobile Phase A | CX-1 pH Gradient Buffer A, 1X (pH 5.6) |
| Mobile Phase B | CX-1 pH Gradient Buffer B, 1X (pH 10.2) |
| Column | Proteomix SCX NP1.7 4.6 × 100 mm |
| Flow Rate | 0.75 mL/min or specified |
| Column Temperature | 30° C. |
| Gradient Range | 10-100% B |
| Gradient Time | 45 min or specified |

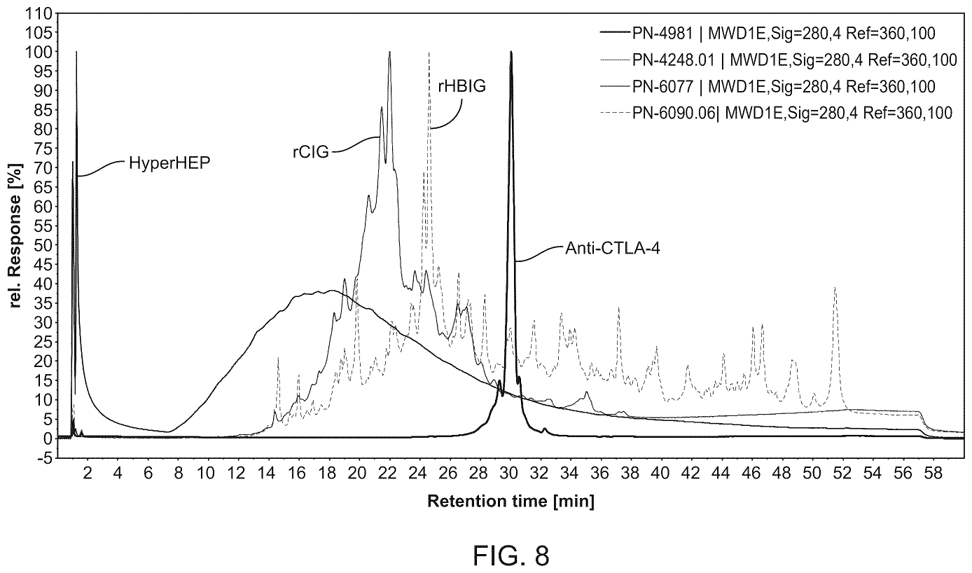

To test specifity of the CEX-HPLC method, the method was performed with different samples-anti-HBV plasma hyperimmune (HyperHEP), a library of ABPs with binding specifity to Hepatitis B virus antigen (rHBIG), a library of ABPs with binding specifity to CoV antigen (rCIG), a recombinant monoclonal antibody (anti-CTLA-4). FIG. 8 provides the results. It shows that the CEX-HPLC chromatograms are specific to each sample and represent their unique characterises. The results suggest that the method can be used to characterize and differentiate different samples.

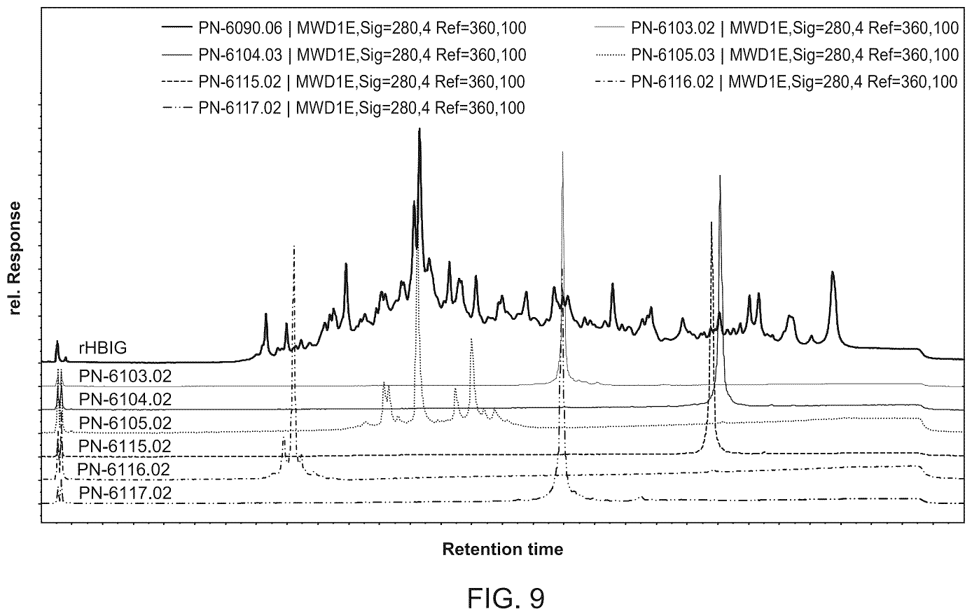

The CEX-HPLC method was performed with the library of ABPs with binding specifity to Hepatitis B virus antigen (rHBIG) and six individual antibodies in the library (PN-6103.02, 6104.02, 6105.02, 6115.02, 6116.02, 6117.02). The results are provided in FIG. 9. The results show that HPLC signals from the rHBIG represent signals from a combination of individual monoclonal antibodies in the library.

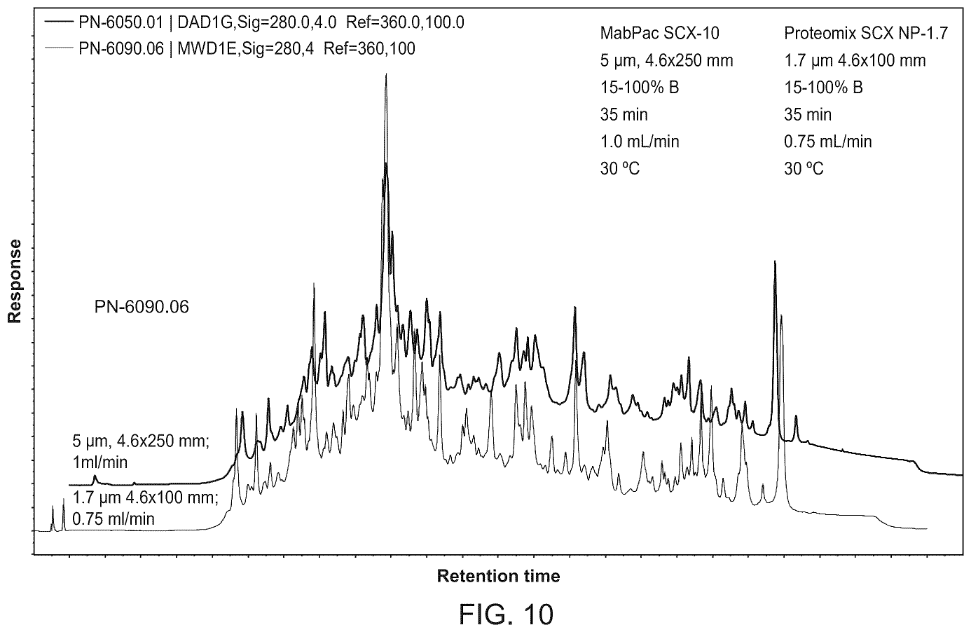

To understand the comparability of the HPLC chromatograms collected using columns of different brands, the rHBIG was analyzed using columns of two different brands under the condition provided below. The results are provided in FIG. 10. They show that HPLC signals from Proteomix SCX NP-1.7 show comparable resolution to the MabPac SCX-10. They had overall similar profiles but had difference in the details when columns from different vendors were used.

HPLC Condition

| | | |
|---|---|---|
| Column | Proteomix SCX | MabPac SCX-10 5 μm, |
| | NP1.7 4.6 × 100 mm | 4.6 × 250 mm |
| Flow Rate | 0.75 mL/min | 1 mL/min |
| Column | 30° C. | 30° C. |
| Temperature | | |
| Gradient Range | 15-100% B | 15-100% B |
| Gradient Time | 35 min | 35 min |

These studies suggest that the CEX-HPLC method can distinguish rHBIG from anti-HBV hyperimmune product (HyperHEP), monoclonal antibody (anti-CTLA-4), or a different recombinant polyclonal antibody (anti-CoV-2; rCIG). The analysis provides reproducible profiles for the same sample in the same method as well as for the same sample from different lots of the same column. These together suggest that the CEX-HPLC method is reliable and suitable for fingerprint type identity test for a library of ABPs.

### 7.1.7. Example 7: Size Heterogeneity by SEC and SEC-MALS

A high-resolution SEC-HPLC method has been developed to characterize the high molecular weight species (HMW), polyclonal antibody monomers (pAb peaks), and low molecular weight species (LMW) in the library of ABPs. This method can be used in process development, formulation development, in-process testing, and release and stability studies.

The high-resolution SEC-HPLC method involves the following steps:
Equipment Setup
Prepare the HPLC system following the system protocol.
Connect to the HPLC column.
Acquisition method parameters:

| Parameters | Setup |
|---|---|
| Injector | Standard injection with needle wash |
| Injection volume | 1.0-3.0 μL |
| Pump flow rate | 0.5-1 mL/min |
| Stop time | 15-30 min |
| Pressure limit | 400 Bar |
| Stepwise gradient | Isocratic 100% Mobile Phase |
| Column temperature | 30° C. |
| Detector signal wavelength | 280 nm with 4 nm bandwidth |
| | 360 nm reference with 100 nm bandwidth |

### Example HPLC Sequence:

Sample Type

Blank (Mobile phase)*
System Suitability
Reference Standard (30 mg/mL)
Blank (Mobile phase)
Test sample 1
. . .
Test sample 5
(add one blank injection between each sample)
Blank (Mobile phase)
Reference Standard (30 mg/mL)
System Suitability
Blank (Mobile phase)
*Slowly increase the flow rate to desired value to avoid rapid pressure increases, which can damage the column. Equilibrate the column for at least 30 minutes at the start of a sequence.
**Include 1 mobile phase blank injection after every 3 sample injections.

The SEC method used herein includes multiple improvements over the traditional processes to achieve a high resolution for analysis of the polyclonal antibodies. A hybrid chemistry called BEH (ethylene bridge hybrid) was employed to reduce residual silanol groups on the stationary phase, the mobile phase (1×PBS) was adjusted to pH 7.0 and the concentration of NaCl was increased to 500 mM (10 mM PBS with 500 mM NaCl, pH7.0) to further reduce charge-based protein column interactions. A column packed with small particle size resin (e.g., with a particle size of less than 2.7 μm) was selected to improve separation efficiency. The column temperature was elevated to 30° C. to reduce the antibody-column interaction. A monoclonal antibody elutes with a narrow main peak on this column. This method can be used for characterizing the size heterogeneity of polyclonal antibodies drug substance, drug product, in-process and stability samples.

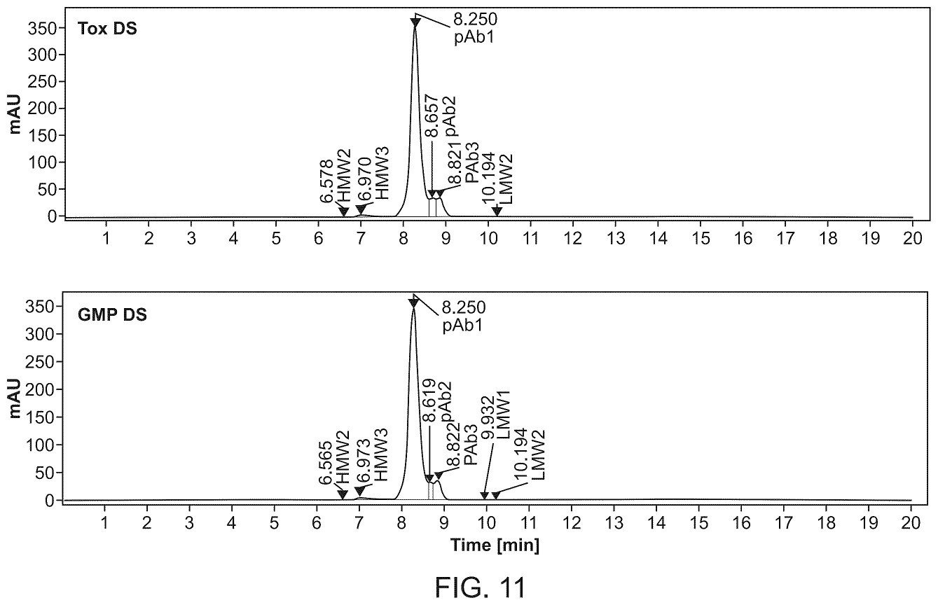

As the library contains more than 1000 antibodies with a diversity of biophysical properties, the presence of multiple monomeric peaks is consistent with the polyclonal nature of the product, as measured in this high-resolution method developed specifically for a polyclonal product. This high-resolution method can distinguish clearly between HMW, polyclonal monomeric peaks, and LMW species present in the library. The Tox and GMP lots had similar profiles (FIG. 11; Table 1) and both had monomeric pAb peaks comprising ~98% and less than ~2% HMW and LMW species overall. Although the instrument software identifies a certain number of peaks within the retention time window that corresponds to antibody monomers, there are actually many more than 2 or 3 overlapping peaks. Resolvable peaks are numbered in the order in which they elute. Due to the overlapping nature of the product profile, some variability in the abundances of individually resolved pAb peaks could be observed.

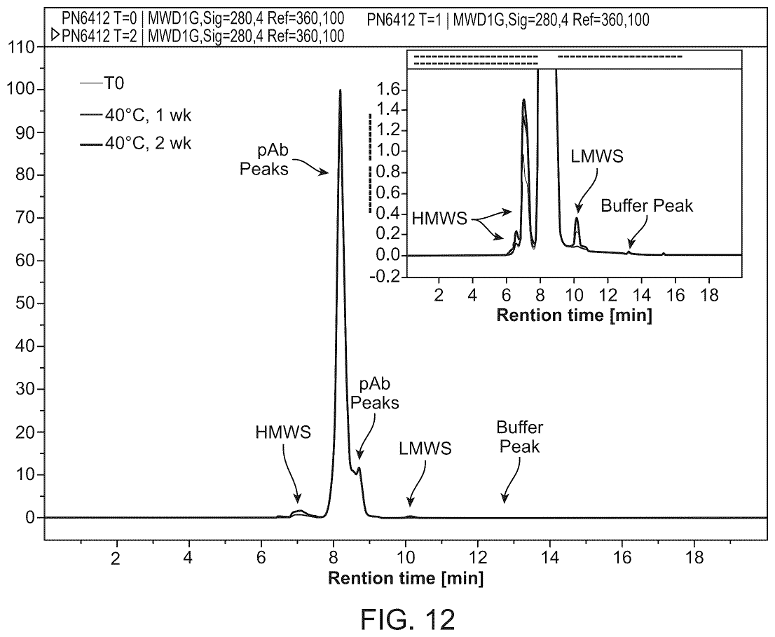

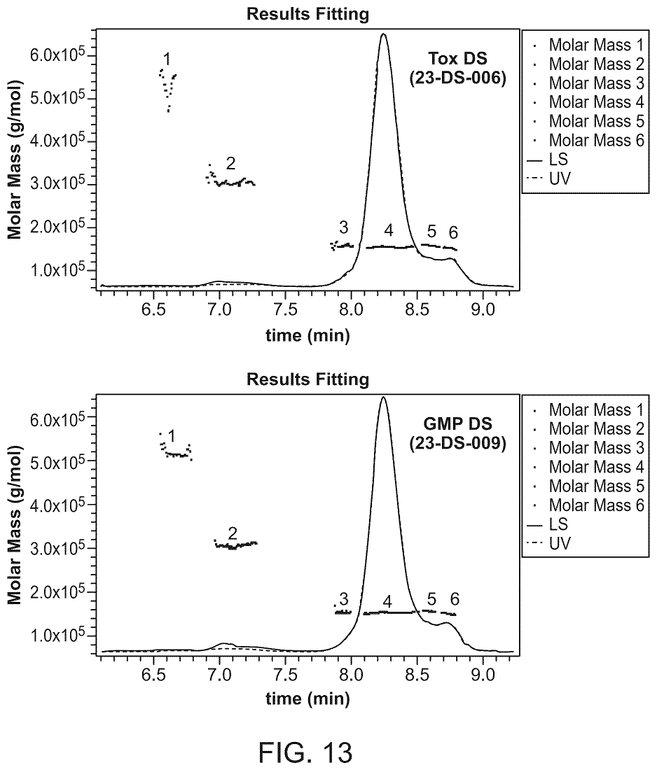

In order to further characterize and qualify the method's ability to resolve HMW and LMW species from monomeric pAb species, a forced degradation study was conducted. A set of example chromatograms of the Tox material incubated at 40° C. for up to 2 weeks are shown in FIG. 12. The chromatograms show that the HMW, LMW, and pAb peaks are well resolved, and that the SEC-HPLC method can detect the increase of LMW species under this stressed condition. The pAb peaks that are clearly distinguished between HMW peaks and LMW peaks have been qualified to elute within the 7.5-9.5 min retention time. The pAb peaks in this qualified retention range (as shown in FIG. 12) are polyclonal antibody monomers (full-length antibodies that are approximately 150 kDa) as confirmed by SEC-multi angle light scattering (MALS). SEC-MALS data also showed comparable results for the Tox and GMP lots (FIG. 13).

The characterization data demonstrate the uniquely developed method's ability to resolve HMW and LMW species from full-length antibody species.

### 7.1.8. Example 8: Size Heterogeneity by CE-SDS

CE-SDS (Capillary electrophoresis sodium dodecyl sulfate) was used as an orthogonal method for characterizing the size heterogeneity of rHBIG in non-reduced and reduced conditions. Briefly, antibody samples were denatured with SDS with or without reducing reagents (Dithiothreitol, DTT), and then introduced electrokinetically into a fused silica capillary filled with a molecular sieving reagent. Once a high voltage electric field was applied, antibody molecules, rendered negatively charged by SDS, were mobilized and resolved according to their sizes. The two key parameters for protein sizing and quantitation were migration time and absorbance. The migration time relates to the size of the proteins: larger proteins move slower due to the sieving effect of the gel inside the chip channels. Absorbance intensity, in turn, is related to the concentration of these proteins. The abundance of a protein is calculated by the percentage of migration time-corrected peak area.

The results show that the method is stability indicating, with % (HC+LC) and % pAb.

### 7.1.9. Example 9: Dynamic Light Scattering (DLS) Characterization

Dynamic light scattering (DLS) is a technique for studying the size heterogeneity of proteins. This technique measures the time dependent intensity of light scattered by diffusing protein particles. From the fluctuating light intensity, an autocorrelation function can be calculated. The shape of the autocorrelation function is correlated with the size and size distribution of the particles in the sample.

### 7.1.10. Example 10: Characterization of Protein Melting Temperature (Tm's)

Melting temperature (Tₘ) is an indication of the thermal stability of protein tertiary structure. High Tₘ is associated with high structural stability. The Tm of rHBIG was tested with two methods, fluorescence-based protein thermal shift (PTS) and differential scanning calorimetry (DSC). In the PTS method, a protein and fluorescent dye mixture was subjected to a thermal ramp from 25° C. to 95° C. Upon protein melting, the interaction between the fluorescent dye and thermally exposed hydrophobic residues enhances the fluorescence. Monitoring the fluorescence intensity as a function of temperature provides the phase change of the protein domains upon temperature increase.

DSC is another technique for characterizing the Tm's of antibodies. Briefly, protein samples were subject to a thermal ramp from 20° C. to 100° C. Heat flow during the temperature ramp was recorded, and heat capacity as a function of temperature was calculated from heat flow after the contribution from formulation buffer was subtracted. Protein melting events manifest as changes in heat capacity.

The results showed that DSC and PTS can be used to probe protein melting events.

### 7.1.11. Example 11: Anticomplement Assay (ACA)

To test the potential for HMW species (specifically aggregates) to trigger non-specific activation of complement, anticomplement assay (ACA) was conducted.

Briefly, immunoglobulin was incubated with guinea pig complement. Non-specific binding of the immunoglobulin preparation to complement led to depletion of complement in the sample. After this incubation step, the sample was titrated with a combination of hemolysin (rabbit antibodies against sheep erythrocytes) and sensitized sheep red blood cells. The sheep red blood cells were sensitized by loading with hemolysin. The extent of lysis of the sheep red blood cells was determined photometrically by measurement of released hemoglobin at 541 nm, and the results were reported in units of complement activity (CH₅₀).

The ACA assay showed a high degree of complexity and potential for variability, due to the biologic reagents used. Therefore, these samples were also tested in a human in vitro system of non-specific complement activation that measures C5a by ELISA after incubation with human serum.

All rHBIG samples tested fell within the Pharm. Eur. Monograph acceptance criteria for IVIG of ≤1 CH₅₀/mg IgG, which does not include any adjustment for the significantly lower dose anticipated for rHBIG, as compared to the dose used for IVIG.

### 8. INCORPORATION BY REFERENCE

This application claims priority to and the benefit of U.S. Provisional Patent Application No. 63/519,815 filed Aug. 15, 2023, the entire contents of which are incorporated by reference herein.

All publications, patents, patent applications and other documents cited in this application are hereby incorporated by reference in their entireties for all purposes to the same extent as if each individual publication, patent, patent application or other document were individually indicated to be incorporated by reference for all purposes.

### 9. EQUIVALENTS

Whereas various specific embodiments have been illustrated and described, the above specification is not restrictive. It will be appreciated that various changes can be made without departing from the spirit and scope of the invention(s). Many variations will become apparent to those skilled in the art upon review of this specification.

1. A method of analyzing a test library comprising antigen binding proteins (ABPs), comprising:
(a) measuring a distribution of charge states of the test library, wherein the test library comprises at least 100 ABPs;
(b) comparing the distribution of charge states with a reference distribution, wherein the reference distribution is a distribution of charge states of a reference library; and
(c) determining a quality of the test library based on the comparison.

2. The method of claim 1, wherein the distribution of charge states is measured in step (a) by cation exchange-High Performance Liquid Chromatography (CEX-HPLC).

3. The method of claim 2, wherein the CEX-HPLC is performed with one or more of:
a pH gradient from a mobile phase A to a mobile phase B, wherein the mobile phase A has a low pH from pH 5 to pH 7 and the mobile phase B has a high pH from pH 9 to pH 11;
a column comprising a strong cation exchanger or a weak cation exchanger;
a flow rate between 0.25 mL/min and 2 mL/min;
at a flow rate between 0.5 mL/min and 1.5 mL/min, between 0.75 mL/min and 1 mL/min, or between 0.75 mL/min and 0.85 mL/min;
a flow rate of 0.5 mL/min, 0.75 mL/min or 1.0 mL/min;
a temperature at 20-40° C., at 25-35° C., at 25° C., at 30° C., or at 35° C.;
5-100% B gradient, 10-100% B gradient, 15-100% B gradient or 20-100% B gradient;
30-60 min gradient time, 30-45 min gradient time, 30-40 min gradient time, or 35 min gradient time, 40 min gradient time, 45 min gradient time, 50 min gradient time, 55 min gradient time, or 60 min gradient time; and
a column packed with a resin having a particle size smaller than 3 μm, smaller than 2.9 μm, smaller than 2.8 μm, smaller than 2.7 μm, smaller than 2.6 μm, or smaller than 2.5 μm, optionally wherein the resin is a porous resin or a non-porous resin.

4-12. (canceled)

13. The method of claim 1, wherein the reference distribution is measured by:
cation exchange-High Performance Liquid Chromatography (CEX-HPLC); or
cation exchange-High Performance Liquid Chromatography (CEX-HPLC) under a condition identical to the condition of measuring the distribution of charge states in (a).

14. (canceled)

15. The method of claim 1, wherein the reference library comprises:
the same ABPs as the test library;
one antibody;
a plurality of antibodies; or
a subset of the at least 100 ABPs in the test library.

16. The method of claim 1, wherein the test library and the reference library are produced from the same production cell line or a progeny thereof, or have been separately generated.

17. The method of claim 1, wherein the reference library:
has been analyzed by sequencing;
is a batch different from the test library; or
has been generated by mixing a plurality of monoclonal antibodies.

18-24. (canceled)

25. The method of claim 1, wherein in step (b), the distribution of charge states is compared with a reference distribution based on peak retention times, peak areas, peak heights, or peak numbers.

26. (canceled)

27. The method of claim 1, wherein in step (c), the test library is determined to have;
a better quality when its distribution of charge states is closer to the reference distribution compared to when its distribution of charge states is further from the reference distribution; or
a good quality when its distribution of charge states is at least 50%, 60%, 70%, 80%, 90%, 96%, 97%, 98%, or 99% identical to the reference distribution in terms of peak areas, peak heights, peak retention times, or peak numbers.

28. (canceled)

29. The method of claim 1, wherein in step (c), the quality of the test library is determined further based on one or more of:
potency of the test library measured by ELISA;
analysis of size heterogeneity of the test library by size exclusion chromatography (SEC)-HPLC.

30-31. (canceled)

32. The method of claim 29, wherein the SEC-HPLC is performed:
with an SEC column comprising small particle resin, wherein the particle size is smaller than 2.9 μm, 2.8 μm, 2.7 μm, 2.6 μm, or 2.5 μm;
with a mobile phase comprising a NaCl concentration between 450 mM and 550 mM, between 480 mM and 520 mM or about 500 mM;
using a BEH stationary phase; or
using a mobile phase with a PH between 6.7 and 7.3, between 6.8 and 7.2, between 6.9 and 7.1 or about 7.0.

33-35. (canceled)

36. The method of claim 1, wherein in step (c), the quality of the test library is determined further based on at least one of followings:
(a) amino acid sequences of IgG1 and IgK framework, optionally verified with an LC-MS reduced peptide mapping method;
(b) a disulfide bond linkage between IgG1 and IgK constant region, optionally verified with an LC-MS non-reduced peptide mapping method;
(c) a size heterogeneity of the test library, optionally characterized by a multi-angle light scattering (MALS) technique;
(d) a melting temperature of the test library, optionally measured by differential scanning calorimetry (DSC);
(e) a glass transition temperature of the test library, optionally measured by differential scanning calorimetry (DSC);
(f) released N-glycan analysis;
(g) total sialic acid quantification; and
(h) hemagglutination assay.

37. The method of claim 1, wherein the test library is a pharmaceutical composition comprising the ABPs and a pharmaceutically acceptable excipient.

38. The method of claim 1, wherein the test library has been prepared by a process comprising:
(a) generation the at least 100 ABPs by culturing a production cell line; and
(b) purification of the at least 100 ABPs.

39. The method of claim 38, wherein the purification is performed by at least one of the following steps:
(i) affinity chromatography,
(ii) low pH virus inactivation,
(iii) hydrophobic interaction chromatography or membrane filtration,
(iv) multimodal anion exchange chromatography or membrane filtration,
(v) multimodal cation exchange chromatography,
(vi) anion exchange chromatography or membrane filtration,
(vii) cation exchange chromatography,
(viii) virus filtration, and
(ix) ultrafiltration and/or diafiltration.

40. (canceled)

41. The method of claim 1, wherein the ABPs are antibodies or antibodies specific to an antigen.

42-43. (canceled)

44. The method of claim 1, further comprising selecting the test library for preparation of a pharmaceutical composition if the test library meets acceptance criteria.

45. The method of claim 44, wherein the test library meets acceptance criteria when one or more factors are met:
a) the test library has charge distribution at least 50%, 60%, 70%, 75%, 80%, 85%, 90%, 96%, 97%, 98%, or 99% identical to the reference distribution in terms of peak sizes, peak retention times, and/or peak numbers;
(b) SEC (size exclusion chromatography)-HPLC of the test library indicates that peaks corresponding to polyclonal antibodies represent more than 65%, 70%, 75%, 80%, 85%, 90%, 95%, 97%, 98%, or 99% of the entire peaks;
(c) SEC (size exclusion chromatography)-HPLC of the test library indicates that peaks corresponding to high molecular weight (HMW) represent less than 5.0%, less than 4.0%, less than 3.0%, less than 2.0% or less than 1.0% of the entire peaks; and
(d) SEC (size exclusion chromatography)-HPLC of the test library indicates that peaks corresponding to low molecular weight (LMW) represent less than 5.0%, less than 4.0%, less than 3.0%, less than 2.0% or less than 1.0% of the entire peaks.

46-48. (canceled)

49. A method of analyzing a test library comprising antigen binding proteins (ABPs), comprising:
(a) measuring size heterogeneity of the test library, wherein the test library comprises at least 100 ABPs;
(b) comparing peaks corresponding to high molecular weight species (HMW), polyclonal antibody monomers (pAb peaks), and low molecular weight species (LMW); and
(c) determining a quality of the test library based on the comparison.

50. The method of claim 49, wherein the size heterogeneity of the test library is measured by Size Exclusion Chromatography (SEC)-HPLC, wherein SEC-HPLC is performed:
with an SEC column comprising a small particle resin, wherein the particle size is smaller than 2.9 μm, 2.8 μm, 2.7 μm, 2.6 μm, or 2.5 μm;
using a BEH stationary phase;
using a mobile phase with a pH between 6.7 and 7.3, between 6.8 and 7.2, between 6.9 and 7.1 or about 7.0;
with a mobile phase with a NaCl concentration between 450 mM and 550 mM, between 480 mM and 520 mM or about 500 mM; or
at a temperature between 28° C. and 32° C., between 29° C. and 31° C. or at about 30° C.

51-55. (canceled)

56. The method of claim 49, wherein the test library meets acceptance criteria when one or more of:
a. SEC (size exclusion chromatography)-HPLC of the test library indicates that peaks corresponding to polyclonal antibodies represent more than 65%, 70%, 75%, 80%, 85%, 90%, 95%, 97%, 98%, or 99% of the entire peaks;
b. SEC (size exclusion chromatography)-HPLC of the test library indicates that peaks corresponding to high molecular weight (HMW) represent less than 5.0%, less than 4.0%, less than 3.0%, less than 2.0% or less than 1.0% of the entire peaks; and/or
c. SEC (size exclusion chromatography)-HPLC of the test library indicates that peaks corresponding to low molecular weight (LMW) represent less than 5.0%, less than 4.0%, less than 3.0%, less than 2.0% or less than 1.0% of the entire peaks,

57. The method of claim 49, further comprising preparing a pharmaceutical composition comprising the test library.

58. A pharmaceutical composition comprising the test library prepared by the method of claim 57.
